(19) United States
(12) Patent Application Publication
(10) Pub. No.: US 2005/0288503 A1
(43) Pub. Date: Dec. 29, 2005

(54) Novel compounds

(76) Inventors: Jerry Leroy Adams, King Of Prussia, PA (US); Jeffrey C. Boehm, King of Prussia, PA (US); Zehong Wan, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/525,478

(22) PCT Filed: Aug. 26, 2003
(86) PCT No.: PCT/US03/26508

§ 371 Date: Feb. 24, 2005

(30) Foreign Application Priority Data
Sep. 6, 2002 (US) 60408832

(51) Int. Cl. C07D 473/10

(52) U.S. Cl. 544/276; 544/277

(57) ABSTRACT

Novel substituted pyrrolo[2,3-d]pyrimidin-4-yl compounds and compositions for use in therapy as CSBP/p38 kinase inhibitors.

### FIELD OF THE INVENTION

This invention relates to a novel group of 7H-pyrrolo[2,3-d]pyrimidine-4-yl ureas and 9H-purin-6-yl ureas compounds, processes for the preparation thereof, the use thereof in treating CSBP/p38 kinase mediated diseases and pharmaceutical compositions for use in such therapy.

### BACKGROUND OF THE INVENTION

Intracellular signal transduction is the means by which cells respond to extracellular stimuli. Regardless of the nature of the cell surface receptor (e.g. protein tyrosine kinase or seven-transmembrane G-protein coupled), protein kinases and phosphatases along with phopholipases are the essential machinery by which the signal is further transmitted within the cell [Marshall, J. C. Cell, 80, 179-278 (1995)]. Protein kinases can be categorized into five classes with the two major classes being, tyrosine kinases and serine/threonine kinases depending upon whether the enzyme phosphorylates its substrate(s) on specific tyrosine(s) or serine/threonine(s) residues [Hunter, T., Methods in Enzymology (Protein Kinase Classification) p. 3, Hunter, T.; Sefton, B. M.; eds. vol. 200, Academic Press; San Diego, 1991].

For most biological responses, multiple intracellular kinases are involved and an individual kinase can be involved in more than one signaling event. These kinases are often cytosolic and can translocate to the nucleus or the ribosomes where they can affect transcriptional and translational events, respectively. The involvement of kinases in transcriptional control is presently much better understood than their effect on translation as illustrated by the studies on growth factor induced signal transduction involving MAP/ERK kinase [Marshall, C. J. Cell, 80, 179 (1995); Herskowitz, I. Cell, 80, 187 (1995); Hunter, T. Cell, 80, 225 (1995); Seger, R., and Krebs, E. G. FASEB J., 726-735 (1995)].

While many signaling pathways are part of cell homeostasis, numerous cytokines (e.g., IL-1 and TNF) and certain other mediators of inflammation (e.g., COX-2, and iNOS) are produced only as a response to stress signals such as bacterial lipopolysaccharide (LPS). The first indications suggesting that the signal transduction pathway leading to LPS-induced cytokine biosynthesis involved protein kinases came from studies of Weinstein [Weinstein, et al., J. Immunol. 151, 3829(1993)] but the specific protein kinases involved were not identified. Working from a similar perspective, Han [Han, et al., Science 265, 808(1994)] identified murine p38 as a kinase which is tyrosine phosphorylated in response to LPS. Definitive proof of the involvement of the p38 kinase in LPS-stimulated signal transduction pathway leading to the initiation of proinflammatory cytokine biosynthesis was provided by the independent discovery of p38 kinase by Lee [Lee; et al., Nature, 372, 739(1994)] as the molecular target for a novel class of anti-inflammatory agents. The discovery of p38 (termed by Lee as CSBP 1 and 2) provided a mechanism of action of a class of anti-inflammatory compounds for which SK&F 86002 was the prototypic example. These compounds inhibited IL-1 and TNF synthesis in human monocytes at concentrations in the low uM range [Lee, et al., Int. J. Imunopharmac. 10(7), 835(1988)] and exhibited activity in animal models which are refractory to cyclooxygenase inhibitors [Lee; et al., Annals N.Y. Acad. Sci., 696, 149(1993)].

It is now firmly established that CSBP/p38 is a one of several kinases involved in a stress-response signal transduction pathway, which is parallel to and largely independent of the analogous mitogen-activated protein kinase (MAP) kinase cascade. Stress signals, including LPS, pro-inflammatory cytokines, oxidants, UV light and osmotic stress, activate kinases upstream from CSBP/p38 which in turn phosphorylate CSBP/p38 at threonine 180 and tyrosine 182 resulting in CSBP/p38 activation. MAPKAP kinase-2 and MAPKAP kinase-3 have been identified as downstream substrates of CSBP/p38 which in turn phosphorylate heat shock protein Hsp 27 (FIG. 1). Additional downstream substrates known to be phosphorylated by p38 include kinases (Mnk1/2, MSK1/2 and PRAK) and transcription factors (CHOP, MEF2, ATF2 and CREB). While many of the signaling pathways required for cytokine biosynthesis remain unknown it appears clear that many of the substrates for p38 listed above are involved. [Cohen, P. Trends Cell Biol., 353-361(1997) and Lee, J. C. et al, Pharmacol. Ther. vol 82, nos 2-3, pp 389-397, 1999].

What is known, however, is that in addition to inhibiting IL-1 and TNF, CSBP/p38 kinase inhibitors (SK&F 86002 and SB 203580) also decrease the synthesis of a wide variety of pro-inflammatory proteins including, IL-6, IL-8, GM-CSF and COX-2. Inhibitors of CSBP/p38 kinase have also been shown to suppress the TNF-induced expression of VCAM-1 on endothelial cells, the TNF-induced phosphorylation and activation of cytosolic PLA2 and the IL-1-stimulated synthesis of collagenase and stromelysin. These and additional data demonstrate that CSBP/p38 is involved not only cytokine synthesis, but also in cytokine signaling [CSBP/P38 kinase reviewed in Cohen, P. Trends Cell Biol., 353-361(1997)].

Interleukin-1 (IL-1) and Tumor Necrosis Factor (END) are biological substances produced by a variety of cells, such as monocytes or macrophages. IL-1 has been demonstrated to mediate a variety of biological activities thought to be important in immunoregulation and other physiological conditions such as inflammation [See, e.g., Dinarello et al., Rev. Infect. Disease, 6, 51 (1984)]. The myriad of known biological activities of IL-1 include the activation of T helper cells, induction of fever, stimulation of prostaglandin or collagenase production, neutrophil chemotaxis, induction of acute phase proteins and the suppression of plasma iron levels.

There are many disease states in which excessive or unregulated IL-1 production is implicated in exacerbating and/or causing the disease. These include rheumatoid arthritis, osteoarthritis, endotoxemia and/or toxic shock syndrome, other acute or chronic inflammatory disease states such as the inflammatory reaction induced by endotoxin or inflammatory bowel disease; tuberculosis, atherosclerosis, muscle degeneration, cachexia, psoriatic arthritis, Reiter's syndrome, rheumatoid arthritis, gout, traumatic arthritis, rubella arthritis, and acute synovitis. Recent evidence also links IL-1 activity to diabetes and pancreatic β cells [review of the biological activities which have been attributed to IL-1 Dinarello, J. Clinical Immunology, 5 (5), 287-297 (1985)].

Excessive or unregulated TNF production has been implicated in mediating or exacerbating a number of diseases including rheumatoid arthritis, rheumatoid spondylitis, osteoarthritis, gouty arthritis and other arthritic conditions; sepsis, septic shock, endotoxic shock, gram negative sepsis, toxic shock syndrome, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, such as influenza, cachexia secondary to infection or malignancy, cachexia, secondary to acquired immune deficiency syndrome (AIDS), AIDS, ARC (AIDS related complex), keloid formation, scar tissue formation, Crohn's disease, ulcerative colitis, or pyresis.

Interleukin-8 (IL-8) is a chemotactic factor produced by several cell types including mononuclear cells, fibroblasts, endothelial cells, and keratinocytes. Its production from endothelial cells is induced by IL-1, TNF, or lipopolysachharide (LPS). IL-8 stimulates a number of functions in vitro. It has been shown to have chemoattractant properties for neutrophils, T-lymphocytes, and basophils. In addition it induces histamine release from basophils from both normal and atopic individuals as well as lysozomal enzyme release and respiratory burst from neutrophils. IL-8 has also been shown to increase the surface expression of Mac-1 (CD11b/CD 18) on neutrophils without de novo protein synthesis, this may contribute to increased adhesion of the neutrophils to vascular endothelial cells. Many diseases are characterized by massive neutrophil infiltration. Conditions associated with an increased in IL-8 production (which is responsible for chemotaxis of neutrophil into the inflammatory site) would benefit by compounds which are suppressive of IL-8 production.

IL-1 and TNF affect a wide variety of cells and tissues and these cytokines as well as other leukocyte derived cytokines are important and critical inflammatory mediators of a wide variety of disease states and conditions. The inhibition of these cytokines is of benefit in controlling, reducing and alleviating many of these disease states.

Inhibition of signal transduction via CSBP/p38, which in addition to IL-1, TNF and IL-8 described above is also required for the synthesis and/or action of several additional pro-inflammatory proteins (i.e., IL-6, GM-CSF, COX-2, collagenase and stromelysin), is expected to be a highly effective mechanism for regulating the excessive and destructive activation of the immune system. This expectation is supported by the potent and diverse anti-inflammatory activities described for CSBP/p38 kinase inhibitors [Badger, et al., J. Pharm. Exp. Thera 279 (3): 1453-1461.(1996); Griswold, et al, Pharmacol. Comm. 7, 323-229 (1996)].

There remains a need for treatment, in this field, for compounds which are cytokine suppressive anti-inflammatory drugs, i.e. compounds which are capable of inhibiting the CSBP/p38/RK kinase.

### BRIEF DESCRIPTION OF THE DRAWINGS

FIG. 1 demonstrates the p38 kinase pathway.

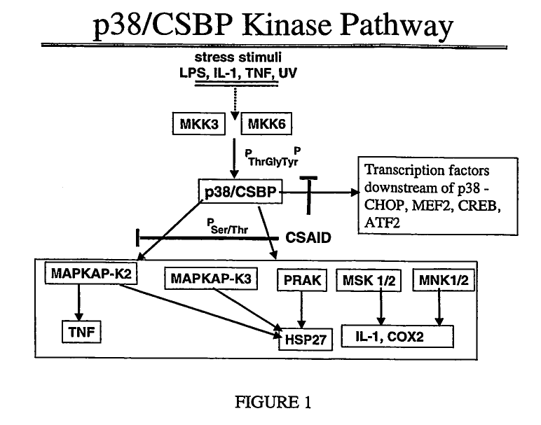

### SUMMARY OF THE INVENTION

This invention relates to the novel compounds of Formula (I), and pharmaceutical compositions comprising a compound of Formula (I), and a pharmaceutically acceptable diluent or carrier.

This invention relates to a method of treating a CSBP/RK/p38 kinase mediated disease in a mammal in need thereof, which comprises administering to said mammal an effective amount of a compound of Formula (I).

This invention also relates to a method of inhibiting cytokines and the treatment of a cytokine mediated disease, in a mammal in need thereof, which comprises administering to said mammal an effective amount of a compound of Formula (I).

This invention more specifically relates to a method of inhibiting the production of IL-1 in a mammal in need thereof which comprises administering to said mammal an effective amount of a compound of Formula (I).

This invention more specifically relates to a method of inhibiting the production of IL-6 in a mammal in need thereof which comprises administering to said mammal an effective amount of a compound of Formula (I).

This invention more specifically relates to a method of inhibiting the production of IL-8 in a mammal in need thereof which comprises administering to said mammal an effective amount of a compound of Formula (I).

This invention more specifically relates to a method of inhibiting the production of TNF in a mammal in need thereof which comprises administering to said mammal an effective amount of a compound of Formula (I).

Accordingly, the present invention provides a compound of Formula (I): 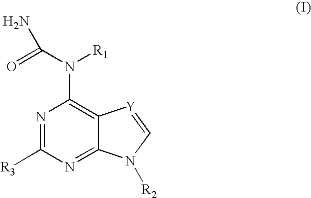
wherein
R₁ is hydrogen, C₁₋₁₀ alkyl, C₃₋₇ cycloalkyl, C₃₋₇ cycloalkyl alkyl, C₅₋₇ cycloalkenyl, C₅₋₇ cycloalkenylalkyl, aryl, arylalkyl, heterocyclic, or heterocyclicalkyl moiety, all of which moieties may be optionally substituted;
R₂ is C₁₋₁₀ alkyl, C₃₋₇ cycloalkyl, C₃₋₇ cycloalkylalkyl, C₅₋₇ cycloalkenyl, C₅₋₇ cycloalkenylalkyl, aryl, arylC₁₋₁₀ alkyl, heteroaryl, heteroarylC₁₋₁₀ alkyl, heterocyclic, heterocyclylC₁₋₁₀ alkyl moiety, all of which moieties may be optionally substituted;
R₃ is an optionally substituted aryl or optionally substituted heteroaryl moiety;
Y is carbon or nitrogen;
   or a pharmaceutically acceptable salt thereof.

### DETAILED DESCRIPTION OF THE INVENTION

The present invention is directed to novel compounds of Formula (I), or compounds of Formula (II), or pharmaceutically acceptable salts thereof.

Compounds of Formula (II) are represented by the structure:
A compound of the formula: 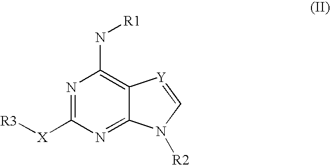
   wherein
R₁ is hydrogen, C₁₋₁₀ alkyl, C₃₋₇ cycloalkyl, C₃₋₇ cycloalkyl alkyl, C₅₋₇ cycloalkenyl, C₅₋₇ cycloalkenylalkyl, aryl, arylalkyl, heterocyclic, heterocyclicalkyl, heteroaryl, or heteroarylalkyl moiety, all of which moieties may be optionally substituted;
R₂ is C₁₋₁₀ alkyl, C₃₋₇ cycloalkyl, C₃₋₇ cycloalkylalkyl, C₅₋₇ cycloalkenyl, C₅₋₇ cycloalkenylalkyl, aryl, arylC₁₋₁₀alkyl, heteroaryl, heteroarylC₁₋₁₀alkyl, heterocyclic, heterocyclylC₁₋₁₀ alkyl moiety, all of which moieties may be optionally substituted;
X is a bond, oxygen, nitrogen or sulfur;
R₃ is an optionally substituted aryl or optionally substituted heteroaryl moiety;
Y is carbon or nitrogen;
   or a pharmaceutically acceptable salt thereof.

While compounds of Formula (II) are utilized herein as a chemical intermediate to make compounds of Formula (I), they have been found to have CSBP inhibitory activity.

For purposes herein, the description of substituent moieties for compounds of Formula (I) also applies to compounds of Formula (II) unless specified otherwise.

Suitably, for compounds of Formula (I) and (II), R₁ is hydrogen, C₁₋₁₀ alkyl, C₃₋₇ cycloalkyl, C₃₋₇ cycloalkylalkyl, C₅₋₇ cycloalkenyl, C₅₋₇ cycloalkenylalkyl, aryl, arylalkyl, heterocyclic, or a heterocyclic alkyl moiety, all of which moieties may be optionally substituted.

The R₁ moieties, including the C₁₋₁₀ alkyl group may be optionally substituted independently by one or more substituents, preferably from one to three substituents, each independently selected from halogen, C₁₋₁₀alkyl, halo-substituted C₁₋₁₀ alkyl, C₂₋₁₀ alkenyl, C₂₋₁₀ alkynyl, C₃₋₇ cycloalkyl, C₃₋₇cycloalkylC₁₋₁₀ alkyl, C₅₋₇ cycloalkenyl, C₅₋₇ cycloalkenyl C₁₋₁₀ alkyl, (CR₁₀R₂₀)ₙOR₆, (CR₁₀R₂₀)ₙSH, (CR₁₀R₂₀)ₙS(O)ₘR₇, (CR₁₀R₂₀)ₙNHS(O)₂R₇, (CR₁₀R₂₀)ₙNR₄R₁₄, (CR₁₀R₂₀)ₙCN, (CR₁₀R₂₀)ₙS(O)₂NR₄R₁₄, (CR₁₀R₂₀)ₙC(Z)R₆, (CR₁₀R₂₀)ₙOC(Z)R₆, (CR₁₀R₂₀)ₙC(Z)OR₆, (CR₁₀R₂₀)ₙC(Z)NR₄R₁₄, (CR₁₀R₂₀)ₙNR₁₀C(Z)R₆, (CR₁₀R₂₀)ₙNR₁₀C(═NR₁₀)NR₄R₁₄, (CR₁₀R₂₀)ₙOC(Z)NR₄R₁₄, (CR₁₀R₂₀)ₙNR₁₀C(Z)NR₄R₁₄, or (CR₁₀R₂₀)ₙNR₁₀C(z)OR₇.

Preferably, R₁ is an optionally substituted aryl, more preferably an optionally substituted phenyl. The aryl ring is preferably substituted, and is more preferably a di-substituted ring system. Suitably, it is substituted in the 4-position or disubstituted in the 2,6 position if phenyl. Preferable substituents include (CR₁₀R₂₀)ₙNR₄R₁₄, (CR₁₀R₂₀)ₙS(O)₂NR₄R₁₄, (CR₁₀R₂₀)ₙNHS(O)₂R₇, or halogen. More preferably the substituents are independently halogen, amine, or di-substituted, such as in 2,6 difluoro or 2,6-dichloro.

Suitably, R₄ and R₁₄ are each independently selected from hydrogen or an optionally substituted C₁₋₄ alkyl, an optionally substituted aryl or an optionally substituted aryl-C₁₋₄ alkyl, or together with the nitrogen which they are attached R₄ and R₁₄ form a heterocyclic ring of 5 to 7 members, which ring optionally contains an additional heteroatom selected from oxygen, sulfur or NR₉.

Suitably, n is 0, or an integer having a value of 1 to 10.

Suitably, m is 0, or the integer 1 or 2.

Suitably, Z is oxygen or sulfur, preferably oxygen.

Suitably, R₆ is hydrogen, C₁₋₁₀alkyl, C₃₋₇ cycloalkyl, heterocyclyl, heterocyclyl C₁₋₁₀alkyl, aryl, arylC₁₋₁₀alkyl, heteroaryl or heteroarylC₁₋₁₀alkyl, wherein these moieties may be optionally substituted.

Suitably, R₇ is C₁₋₆alkyl, aryl, arylC₁₋₆alkyl, heterocyclic, heterocyclylC₁₋₆ alkyl, heteroaryl, or heteroarylC₁₋₆alkyl; and wherein each of these moieties may be optionally substituted.

Suitably, R₉ is hydrogen, C(Z)R₆ or optionally substituted C₁₋₁₀alkyl, optionally substituted aryl or optionally substituted aryl-C₁₋₄ alkyl.

Suitably, R₁₀ and R₂₀ are each independently selected from hydrogen or C₁₋₄ alkyl.

Suitably, for compounds of Formula (I) and (II), R₂ is C₁₋₁₀ alkyl, C₃₋₇ cycloalkyl, C₃₋₇ cycloalkylalkyl, C₅₋₇ cycloalkenyl, C₅₋₇ cycloalkenylalkyl, aryl, arylC₁₋₁₀alkyl, heteroaryl, heteroarylC₁₋₁₀alkyl, heterocyclic, heterocyclylC₁₋₁₀alkyl moiety, all of which moieties may be optionally substituted. Preferably R₂ is an optionally substituted C₁₋₁₀ alkyl, arylC₁₀ alkyl, or heterocyclylC₁₋₁₀ alkyl.

The R₂ moieties, including C₁₋₁₀ alkyl, may be optionally substituted independently by one or more substituents, preferably from one to four substituents each independently selected from halogen, C₁₋₁₀ alkyl, halo-substituted C₁₋₁₀ alkyl, C₂₋₁₀ alkenyl, C₂₋₁₀ alkyl, C₃₋₇ cycloalkyl, C₃₋₇cycloalkylC₁₋₁₀ alkyl, C₅₋₇ cycloalkenyl, C₅₋₇ cycloalkenyl C₁₋₁₀ alkyl, (CR₁₀R₂₀)ₙOR₆, (CR₁₀R₂₀)ₙSH, (CR₁₀R₂₀)ₙS(O)ₘR₇, (CR₁₀R₂₀)ₙNHS(O)₂R₇, (CR₁₀R₂₀)ₙNR₄R₁₄, (CR₁₀R₂₀)ₙCN, (CR₁₀R₂₀)ₙ S(O)₂NR₄R₁₄, (CR₁₀R₂₀)ₙC(Z)R₆, (CR₁₀R₂₀)ₙOC(Z)R₆, (CR₁₀R₂₀)ₙC(Z)OR₆, (CR₁ OR₂₀)ₙC(Z)NR₄R₁₄, (CR₁₀R₂₀)ₙOC(Z)R₆, (CR₁₀R₂₀)ₙNR₁₀C(═NR₁₀)NR₄R₁₄, (CR₁₀R₂₀)ₙOC(Z)NR₄R₁₄, (CR₁₀R₂₀)ₙNR₁₀C(Z)NR₄R₁₄, or (CR₁₀R₂₀)ₙNR₁₀C(Z)OR₇.

Suitably, Y is carbon or nitrogen.

In compounds of Formula (I) and (II), suitably R₃ is an optionally substituted aryl or optionally substituted heteroaryl moiety. Preferably, R₃ is an optionally substituted aryl, more preferably an optionally substituted phenyl.

The R₃ moieties may be optionally substituted one or more times, preferably one to four times, independently by halogen, C₁₋₄ alkyl, halo-substituted-C₁₋₄ alkyl, cyano, nitro, (CR₁₀R₂₀)ᵥNR₄R₁₄, (CR₁₀R₂₀)ᵥC(Z)NR₄R₁₄, (CR₁₀R₂₀)ᵥC(Z)OR₈, (CR₁₀R₂₀)ᵥCORₐ, SR₅, S(O)R₅, S(O)₂R₅, (CR₁₀R₂₀)ᵥOR₈, ZC(Z)R₁₁, NR₁₀C(Z)R₁₁, or NR₁₀S(O)₂R₇.

Preferable the phenyl ring is substituted independently in the 4-position, or di-substituted in the 2,6 position. Suitably substituents include halogen, or alkyl.

Suitably, v is 0, or an integer having a value of 1 or 2.

Suitably, Rₐ is hydrogen, C₁₋₄ alkyl, halo-substituted C₁₋₄ alkyl, C₂₋₄ alkenyl, C₂₋₄ alkynyl, C₃₋₇ cycloalkyl, C₅₋₇ cycloalkenyl, aryl, arylC₁₋₄ alkyl, heteroaryl, heteroarylC₁₋₄ alkyl, heterocyclyl, heterocyclylC₁₋₄ alkyl, (CR₁₀R₂₀)ᵥOR₇, (CR₁₀R₂₀)ᵥS(O)ₘR₇, (CR₁₀R₂₀)ᵥNHS(O)₂R₇, or (CR₁₀R₂₀)ᵥNR₄R₁₄; and wherein the aryl, arylalkyl, heteroaryl, heteroaryl alkyl may be optionally substituted.

Suitably, R₅ is hydrogen, C₁₋₄ alkyl, C₂₋₄ alkenyl, C₂₋₄ alkynyl or NR₄R₁₄, excluding the moieties SR₅ being SNR₄R₁₄, S(O)₂R₅ being SO₂H and S(O)R₅ being SOH.

Suitably, R₈ is hydrogen, C₁₋₄ alkyl, halo-substituted C₂₋₄ alkyl, C₂₋₄ alkenyl, C₂₋₄ alkynyl, C₃₋₇ cycloalkyl, C₅₋₇ cycloalkenyl, aryl, arylC₁₋₄ alkyl, heteroaryl, heteroarylC₁₋₁₄ alkyl, heterocyclyl, heterocyclylC₁₋₄ alkyl, (CR₁₀R₂₀)ₜOR₇, (CR₁₀R₂₀)ₜS(O)ₘR₇, (CR₁₀R₂₀)ₜNHS(O)₂R₇, or (CR₁₀R₂₀)ₜNR₄R₁₄; and wherein the aryl, arylalkyl, heteroaryl, heteroaryl alkyl may be optionally substituted.

Suitably, t is an integer having a value of 1 to 3.

Suitably, R₁₁ is C₁₋₄ alkyl, halo-substituted C₁₋₄ alkyl, C₂₋₄ alkenyl, C₂₋₄ alkyl, C₃₋₇ cycloalkyl, C₅₋₇ cycloalkenyl, aryl, arylC₁₋₄ alkyl, heteroaryl, heteroarylC₁₋₁₄ alkyl, heterocyclyl, heterocyclylC₁₋₄ alkyl, (CR₁₀R₂₀)ₜOR₇, (CR₁₀R₂₀)ₜS(O)ₘR₇, (CR₁₀R₂₀)ₜNHS(O)₂R₇, or (CR₁₀R₂₀)ᵥNR₄R₁₄; and wherein the aryl, arylalkyl, heteroaryl, and heteroaryl alkyl moieties may be optionally substituted.

As used herein, “optionally substituted” unless specifically defined shall mean such groups as halogen, such as fluorine, chlorine, bromine or iodine; hydroxy; hydroxy substituted C₁₋₁₀alkyl; C₁₋₁₀alkoxy, such as methoxy or ethoxy; halosubstituted C₁₋₁₀alkoxy; S(O)m alkyl, such as methyl thio, methylsulfinyl or methyl sulfonyl; NR₄R₁₄, such as amino or mono or -disubstituted C₁₋₄ alkyl or wherein the R₄R₁₄ can cyclize together with the nitrogen to which they are attached to form a 5 to 7 membered ring which optionally contains an additional heteroatom selected from O/N/S; C₁₋₁₀alkyl, C₃₋₇cycloalkyl, or C₃₋₇cycloalkyl C₁₋₁₀alkyl group, such as methyl, ethyl, propyl, isopropyl, t-butyl; etc. or cyclopropyl methyl; halosubstituted C₁₋₁₀alkyl, such CF₂CF₂H, or CF₃; an optionally substituted aryl, such as phenyl, or an optionally substituted arylalkyl, such as benzyl or phenethyl, wherein these aryl containing moieties may also be substituted one to two times by halogen; hydroxy; hydroxy substituted alkyl; C₁₋₁₀alkoxy; S(O)ₘ alkyl; amino, mono & di-substituted C₁₋₄ alkyl amino, such as in the NR₄R₁₄ group; C₁₋₄ alkyl, or CF₃.

Suitable pharmaceutically acceptable salts are well known to those skilled in the art and include basic salts of inorganic and organic acids, such as hydrochloric acid, hydrobromic acid, sulphuric acid, phosphoric acid, methane sulphonic acid, ethane sulphonic acid, acetic acid, malic acid, tartaric acid, citric acid, lactic acid, oxalic acid, succinic acid, fumaric acid, maleic acid, benzoic acid, salicylic acid, phenylacetic acid and mandelic acid.

In addition, pharmaceutically acceptable salts of compounds of Formula (I) may also be formed with a pharmaceutically acceptable cation, for instance, if a substituent group comprises a carboxy moiety. Suitable pharmaceutically acceptable cations are well known to those skilled in the art and include alkaline, alkaline earth, ammonium and quaternary ammonium cations.

The term “halo” or “halogens” is used herein to mean the halogens, chloro, fluoro, bromo and iodo.

The term “C₁₋₁₀alkyl” or “alkyl” or “alkyl₁₋₁₀” is used herein to mean both straight and branched chain radicals of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.

The term “cycloalkyl” is used herein to mean cyclic radicals, preferably of 3 to 8 carbons, including but not limited to cyclopropyl, cyclopentyl, cyclohexyl, and the like.

The term “cycloalkenyl” is used herein to mean cyclic radicals, preferably of 5 to 8 carbons, which have at least one bond including but not limited to cyclopentenyl, cyclohexenyl, and the like.

The term “alkenyl” is used herein at all occurrences to mean straight or branched chain radical of 2-10 carbon atoms, unless the chain length is limited thereto, including, but not limited to ethenyl, 1-propenyl, 2-propenyl, 2-methyl-1-propenyl, 1-butenyl, 2-butenyl and the like.

The term “aryl” is used herein to mean phenyl and naphthyl.

The term “heteroaryl” (on its own or in any combination, such as “heteroaryloxy”, or “heteroaryl alkyl”) is used herein to mean a 5-10 membered aromatic ring system in which one or more rings contain one or more heteroatoms selected from the group consisting of N, O or S, such as, but not limited, to pyrrole, pyrazole, furan, thiophene, quinoline, isoquinoline, quinazolinyl, pyridine, pyrimidine, oxazole, thiazole, thiadiazole, tetrazole, triazole, imidazole, or benzimidazole.

The term “heterocyclic” (on its own or in any combination, such as “heterocyclylalkyl”) is used herein to mean a saturated or partially unsaturated 4-10 membered ring system in which one or more rings contain one or more heteroatoms selected from the group consisting of N, O, or S; such as, but not limited to, pyrrolidine, piperidine, piperazine, morpholine, tetrahydropyran, or imidazolidine.

The term “aralkyl” or “heteroarylalkyl” or “heterocyclicalkyl” is used herein to mean C₁₋₄ alkyl as defined above attached to an aryl, heteroaryl or heterocyclic moiety as also defined herein unless otherwise indicate.

The term “sulfinyl” is used herein to mean the oxide S(O) of the corresponding sulfide, the term “thio” refers to the sulfide, and the term “sulfonyl” refers to the fully oxidized S(O)₂ moiety.

The term “aroyl” is used herein to mean C(O)Ar, wherein Ar is as phenyl, naphthyl, or aryl alkyl derivative such as defined above, such group include but are not limited to benzyl and phenethyl.

The term “alkanoyl” is used herein to mean C(O)C₁₋₁₀alkyl wherein the alkyl is as defined above.

It is recognized that the compounds of the present invention may exist as stereoisomers, regioisomers, or diastereiomers. These compounds may contain one or more asymmetric carbon atoms and may exist in racemic and optically active forms. All of these compounds are included within the scope of the present invention.

Exemplified compounds of Formula (I), include: 1-(2,6-Difluorophenyl)-1-[2-(4-fluoro-2-methylphenyl)-7-methyl-7H-pyrrolo[2,3-d]pyrimidin-4-yl]-3-urea; or a pharmaceutically acceptable salt thereof.

Other exemplified compounds of Formula (I) include the compounds noted below wherein R3 is a phenyl substituted by W, and represented by Formula (Ia):

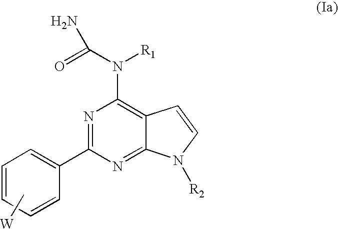

wherein

| R₁ | R₂ | W |
|---|---|---|
| 2,4-diF-phenyl | CH₃ | H |
| CH₂CH₂N(Me)₂ | CH₂CH₂OH | 2,4-di-F |
| CH₂CH₂N-morpholinyl | CH₂CH₂CO2Me | 4-F |
| (R,S)-2-pheneth-1-yl | CH₂CH₂CO2H | 2-Me-4-F |
| 2,6-diF-phenyl | CH₂CH₂NMe₂ | |
| 2,6-diCl-phenyl | CH₂CH₂-imidazol-3-yl | |

or pharmaceutically acceptable salts thereof.

The compounds of Formula (I) and (II) may be obtained by applying synthetic procedures, described herein. The synthesis provided for is applicable to producing compounds of Formula (I) having a variety of different R₁, and R₂, groups which are reacted, employing optional substituents which are suitably protected, to achieve compatibility with the reactions outlined herein. Subsequent deprotection, in those cases, then affords compounds of the nature generally disclosed.

Once the nucleus has been established, further compounds of Formula (I) may be prepared by applying standard techniques for functional group interconversion, well known in the art. For instance: C(O)NR₄R₁₄ from CO₂CH₃ by heating with or without catalytic metal cyanide, e.g. NaCN, and HNR₄R₁₄ in CH₃OH; OC(O)R₃ from OH with e.g., ClC(O)R₃ in pyridine; NR₁₀—C(S)NR₄R₁₄ from NHR₁₀ with an alkylisothiocyante or thiocyanic acid; NR₁₀C(O)OR₇ from NHR₁₀ with the alkyl chloroformate; NR₁₀C(O)NR₄R₁₄ from NHR₁₀ by treatment with an isocyanate, e.g. HN═C═O or R₁₀N═C═O; NR₁₀—C(O)R₇ from NHR₁₀ by treatment with Cl—C(O)R₇ in pyridine; C(═NR₁₀)NR₄R₁₄ from C(NR₄R₁₄)SR₃ with H₃NR₃⁺OAc⁻ by heating in alcohol; C(NR₄R₁₄)SR₃ from C(S)NR₄R₁₄ with R₆—I in an inert solvent, e.g. acetone; C(S)NR₄R₁₄ (where R₄ or R₁₄ is not hydrogen) from C(S)NH₂ with HNR₄R₁₄—C(═NCN)—NR₄R₁₄ from C(═NR₄R₁₄)—SR₃ with NH₂CN by heating in anhydrous alcohol, alternatively from C(═NH)—NR₄R₁₄ by treatment with BrCN and NaOEt in EtOH; NR₁₀—C(═NCN)SR₈ from NHR₁₀ by treatment with (R₈S)₂C═NCN; NR₁₀SO₂R₃ from NHR₁₀ by treatment with ClSO₂R₃ by heating in pyridine; NR₁₀C(S)R₆ from NR₁₀C(O)R₆ by treatment with Lawesson's reagent [2,4-bis(4-methoxyphenyl)-1,3,2,4-dithiadiphosphetane-2,4-disulfide]; NR₁₀SO₂CF₃ from NHR₆ with triflic anhydride and base wherein R₃, R₆, R₇, R₁₀, R₄ and R₁₄ are as defined in Formula (I) herein.

Precursors of the groups R₁, and R₂ can be other R₁, and R₂ groups which can be interconverted by applying standard techniques for functional group interconversion. For example wherein a moiety is a halo substituted C₁₋₁₀alkyl can be converted to the corresponding C₁₋₁₀ alkylN₃ derivative by reacting with a suitable azide salt, and thereafter if desired can be reduced to the corresponding C₁₋₁₀alkylNH₂ compound, which in turn can be reacted with R₇S(O)₂X wherein X is halo (e.g., chloro) to yield the corresponding C₁₋₁₀-alkylNHS(O)₂R₇ compound.

Alternatively wherein the moiety is a halo-substituted C₁₋₁₀-alkyl it can be reacted with an amine R₄R₁₄NH to yield the corresponding C₁₋₁₀-alkylNR₁₄ compound, or can be reacted with an alkali metal salt of R₇SH to yield the corresponding C₁₋₁₀alkylSR₇ compound.

Suitable protecting groups for use with hydroxyl groups and nitrogen groups are well known in the art and described in many references, for instance, Protecting Groups in Organic Synthesis, Greene T W, Wiley-Interscience, New York, 1981. Suitable examples of hydroxylprotecting groups include silyl ethers, such as t-butyldimethyl or t-butyldiphenyl, and alkyl ethers, such as methyl connected by an alkyl chain of variable link, (CR₁₀R₂₀)ₙ.

Pharmaceutically acid addition salts of compounds of Formula (I) may be obtained in known manner, for example by treatment thereof with an appropriate amount of acid in the presence of a suitable solvent.

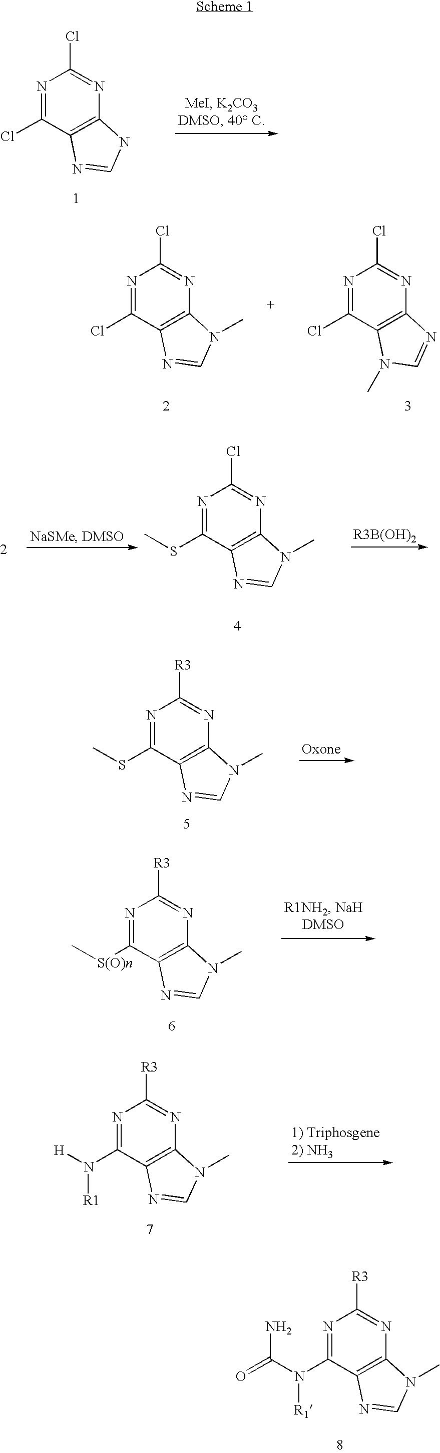

The first strategy for the preparation of compounds of formula (I) is shown in Scheme 1. Methylation of 2,6-dichloropurine (1) provided 2,6-dichloro-9-methyl-9H-purine (2). The literature reports for the synthesis of 2 from 1 describe methods that result in poor yield and poor selectivity due to the formation of another isomer 2,6-dichloro-7-methyl-9H-purine (3) [see Beaman, A. G. et al., J. Org. Chem., 1963, 28, 2310-2313; and Parker, C. W. et al., Phytochemistry, 1986, 25, 3030-310]. However, use of DMSO as the solvent significantly improved the yield of 2 to 60% with 3 formed in small amount (<10%). Selective displacement of the chloride at C-6 of 2 with O, S and N nucleophiles as illustrated for thiolate affords the 6-sulfanyl substituted purines (4). Palladium catalyzed Suzuki coupling with aryl boronic acid proceeds in good to excellent yield to produce 5. Additionally, the biaryl coupling reaction of 4 can be performed using aryl or heteroaryl organozinc, organocopper, organotin, or other organometallic reagents known to afford biaryl cross-coupling products. Oxidation of the sulfide (5) provides either the sulfone or the sulfone plus sulfoxide (6), both of which are suitably activated for displacement with nucleophiles. Thus the activated purines (6) may be reacted with anilines or alkyl amines at either room temperature or 100° C. depending upon the reactivity of the amine affords the secondary amine (7). Reaction of 7 with triphosgene or phosgene or with other activated carbonated equivalents (for example, diphenyl carbonate), followed by treatment with ammonia produces the desired urea (8).

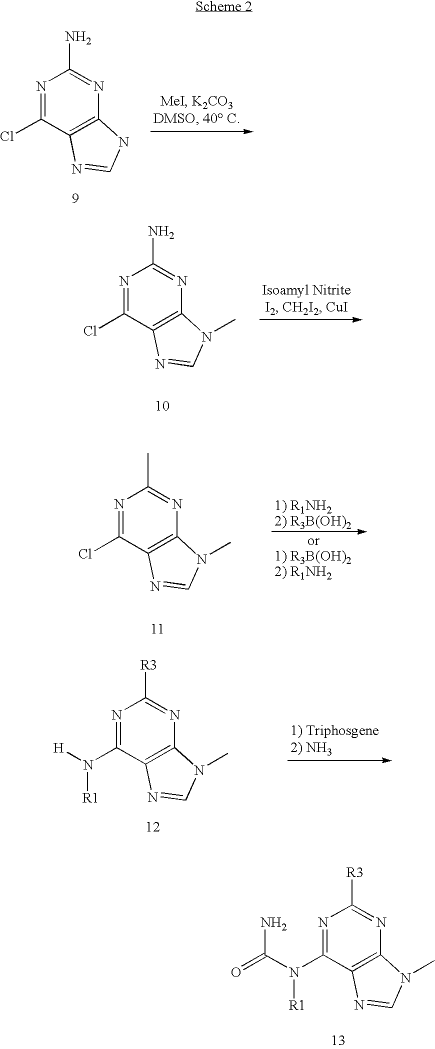

A second strategy for the preparation of compounds of formula (I) is shown in Scheme 2. 6-Chloro-2-iodo-9-methylpurine (11) has been prepared from 2-amino-6-chloropurine (9) by following a two-step literature sequence, methylation with methyl iodide [see Schultz, P. G. et al., J. Am. Chem. Soc. 1996, 118, 7430-7431] and diazotization-substitution with isoamylnitrite and a mixture of CH₂I₂, I₂ and CuI as the iodide source [see Favaudon, V. et al., Bioorg. Med. Chem. 1999, 7, 1281-1293]. Selective displacement of the chloride at C-6 with O, S and N nucleophiles followed by palladium promoted Suzuki coupling with aryl boronic acid furnishes 2,6,9-trisubstituted purines (12). Alternatively the two steps can be switched to the sequence of Suzuki cross coupling followed by displacement of the chloride at C-6 with nucleophiles. Additionally, the biaryl coupling reaction can be performed using aryl or heteroaryl organozinc, organocopper, organotin, or other 5 organometallic reagents known to afford biaryl cross-coupling products. Reaction of 12 with triphosgene or phosgene or with other activated carbonated equivalents (for example, diphenyl carbonate), followed by treatment with ammonia produces the desired urea (13).

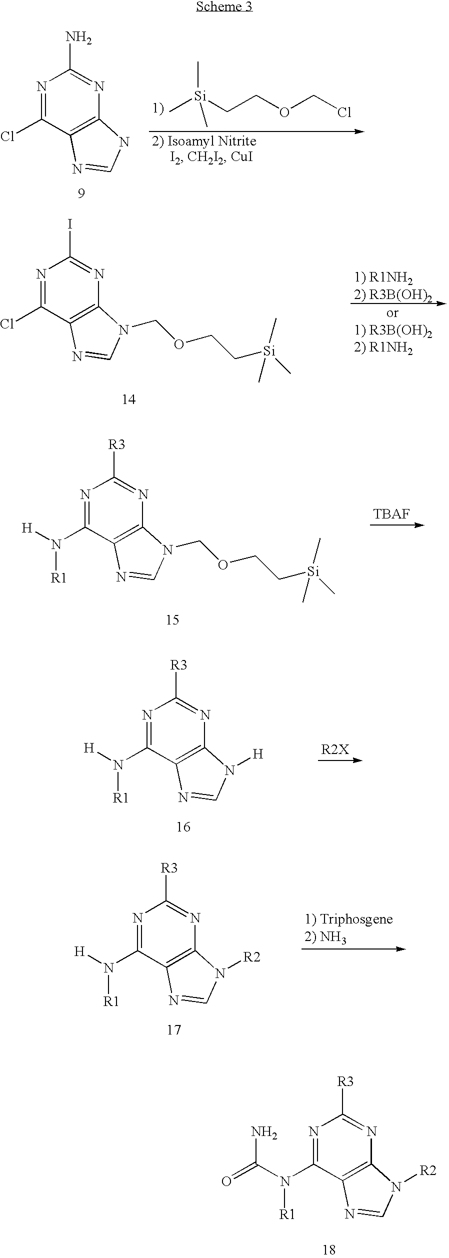

A third strategy for the preparation of compounds of formula (I) is shown in Scheme 3. Selective protection of N-9 of 2-amino-6-chloropurine (9) with easily removable protecting groups, for example benzyl, methoxy-substituted benzyl, methoxymethyl, ter-butyldimethylsilyl, Alloc, Boc and trimethylsilylethoxymethyl followed by diazotization with isoamylnitrite and substitution with a mixture of CH₂I₂, I₂ and CuI as the iodide source provides trisubstituted purine 14. Selective displacement of the chloride at C-6 with O, S and N nucleophiles followed by palladium promoted Suzuki coupling with aryl boronic acid affords 2,6,9-trisubstituted purines (15). Alternatively the two steps can be switched to the sequence of Suzuki cross coupling followed by displacement of the chloride at C-6 with nucleophiles. Additionally, the biaryl coupling reaction can be performed using aryl or heteroaryl organozinc, organocopper, organotin, or other organometallic reagents known to afford biaryl cross-coupling products. Deprotection and alkylation then produce 16. In the electrophile (R₂X), X could be chloride, bromide, iodide, mesylate or tosylate. Reaction of 17 with triphosgene or phosgene or with other activated carbonated equivalents (for example, diphenyl carbonate), followed by treatment with ammonia produces the desired urea (18).

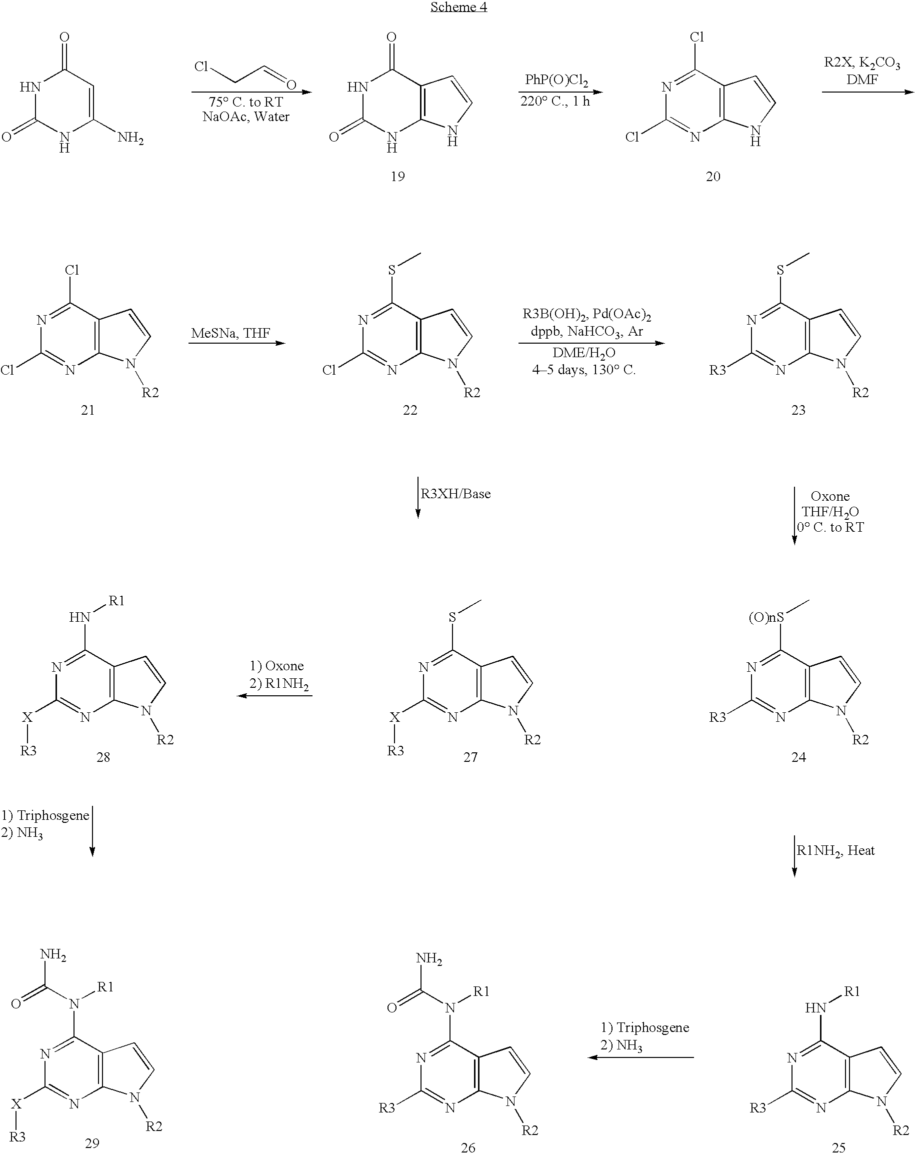

Reaction of 6-aminouracil with chloroacetaldehyde by the literature procedure afforded 1,7-dihydropyrrolo[2,3-d]pyrimidine-2,4-dione (19, Scheme 4) [see Senda, S. et al., Chem. Pharm. Bull 1974, 22(7), 1459-1467]. The literature reports for the synthesis of 2,4-dichloro-7H-pyrrolo[2,3-d]pyrimidine (20) from the dione describe methods that result in capricious yields of 25%. [see Kazimierczuk, et al., J. Am. Chem. Soc. 1984, 106, 6379-6382; and Saxena, et al., J. Med. Chem. 1988, 31, 1501-1506]. However, the use of phenylphosphonic dichloride has been reported to effect good yields of chlorodehydroxylation product in a related heterocycle, when the use of POCl₃-PCl₅ proceeded poorly [Walford, et al., J. Med. Chem. 1971, 12(4), 3]. Application of the former reagent to the present system resulted in a significant improvement in the isolated yields of 20. Furthermore, the reaction was readily scaled up to provide useful quantities of the key intermediate.

Reaction of the dichloro compound (20) with electrophiles by standard methods afforded the 7-alkylated, or acylated intermediates (21). Included among these analogs are compounds containing R₂ that may act as easily removable protecting groups, for example benzyl, methoxy-substituted benzyl, methoxymethyl and trimethylsilylethoxymethyl. Displacement of the chlorides with O,S and N nucleophiles occurrs selectivley at CA and as illustrated for thiolate affords the 4-sulfanyl substituted 1,7-pyrrolo[2,3-d]pyrimidine (22). The Suzuki reaction of 22 with aryl boronic acids using a palladium catalyst, such as, tetrakis (triphenyl-phophine) palladium(0) catalyst proceeds in good to excellent yield to produce 23. Additionally, the biaryl coupling reaction of (22) can be performed using aryl or heteroaryl organozinc, organcopper, organotin, or other organometallic reagents known to afford biaryl cross-coupling products. Alternatively the displacement can be effected with amines or alcohols to produce the R₃—X— linked compounds (27) where X is N, or O. Oxidation of the sulfide (23) with with peracids or Oxone® produces either the sulfone or the sulfone plus sulfoxide (24), both of which are suitably activated for displacement with nucleophiles. Thus the activated pyrimidines (24) may be reacted with anilines or alkyl amines at either room tempertuate depending upon the reactivity of the amine produces the secondary amine (25). For less reactive amines the addition of a strong non-nucleophilic base, such as diisopropyl ethylamine or tetramethylpiperidine may be desirable to increase the reaction rate. For particularly unreactive amines it may be first necessary to form the amine salt with a metal hydride or other strong base which is then added to compound (24) either at room temperature or with heating and using ethereal or diplolar aprotic solvents (DMF, DMSO). Reaction of (25) with tri-phosgene or phosgene or with other activated carbonate equivalents (for example, diphenyl carbonate), followed by treatment with ammonia affords the urea target molecules (26). Alternatively, treatment of the secondary amine (25) with chlorosulfonyl isocyanate may also produce the desired ureas (26). Compound 27, may also be converted to the urea product 29, using the sequence outlined above, for converting 23 to 26.

Methods of Treatment

The compounds of Formula (I) or a pharmaceutically acceptable salt thereof can be used in the manufacture of a medicament for the prophylactic or therapeutic treatment of any disease state in a human, or other mammal, which is exacerbated or caused by excessive or unregulated cytokine production by such mammal's cell, such as but not limited to monocytes and/or macrophages.

Compounds of Formula (I) are capable of inhibiting proinflammatory cytokines, such as IL-1, IL-6, IL-8, and TNF and are therefore of use in therapy. IL-1, IL-6, IL-8 and TNF affect a wide variety of cells and tissues and these cytokines, as well as other leukocyte-derived cytokines, are important and critical inflammatory mediators of a wide variety of disease states and conditions. The inhibition of these pro-inflammatory cytokines is of benefit in controlling, reducing and alleviating many of these disease states.

Accordingly, the present invention provides a method of treating a cytokine-mediated disease which comprises administering an effective cytokine-interfering amount of a compound of Formula (I) or a pharmaceutically acceptable salt thereof.

Compounds of Formula (I) are capable of inhibiting inducible proinflammatory proteins, such as COX-2, also referred to by many other names such as prostaglandin endoperoxide synthase-2 (PGHS-2) and are therefore of use in therapy. These proinflammatory lipid mediators of the cyclooxygenase (CO) pathway are produced by the inducible COX-2 enzyme. Regulation, therefore of COX-2 which is responsible for the these products derived from arachidonic acid, such as prostaglandins affect a wide variety of cells and tissues are important and critical inflammatory mediators of a wide variety of disease states and conditions. Expression of COX-1 is not effected by compounds of Formula (I). This selective inhibition of COX-2 may alleviate or spare ulcerogenic liability associated with inhibition of COX-1 thereby inhibiting prostoglandins essential for cytoprotective effects. Thus inhibition of these pro-inflammatory mediators is of benefit in controlling, reducing and alleviating many of these disease states. Most notably these inflammatory mediators, in particular prostaglandins, have been implicated in pain, such as in the sensitization of pain receptors, or edema. This aspect of pain management therefore includes treatment of neuromuscular pain, headache, cancer pain, and arthritis pain. Compounds of Formula (I) or a pharmaceutically acceptable salt thereof, are of use in the prophylaxis or therapy in a human, or other mammal, by inhibition of the synthesis of the COX-2 enzyme.

Accordingly, the present invention provides a method of inhibiting the synthesis of COX-2 which comprises administering an effective amount of a compound of Formula (I) or a pharmaceutically acceptable salt thereof. The present invention also provides for a method of prophylaxis treatment in a human, or other mammal, by inhibition of the synthesis of the COX-2 enzyme.

In particular, compounds of Formula (I) or a pharmaceutically acceptable salt thereof are of use in the prophylaxis or therapy of any disease state in a human, or other mammal, which is exacerbated by or caused by excessive or unregulated IL-1, IL-6, IL-8 or TNF production by such mammal's cell, such as, but not limited to, monocytes and/or macrophages.

Accordingly, in another aspect, this invention relates to a method of inhibiting the production of IL-1 in a mammal in need thereof which comprises administering to said mammal an effective amount of a compound of Formula (I) or a pharmaceutically acceptable salt thereof.

There are many disease states in which excessive or unregulated IL-1 production is implicated in exacerbating and/or causing the disease. These include-rheumatoid arthritis, osteoarthritis, meningitis, ischemic and hemorrhagic stroke, neurotrauma/closed head injury, stroke, endotoxemia and/or toxic shock syndrome, other acute or chronic inflammatory disease states such as the inflammatory reaction induced by endotoxin or inflammatory bowel disease, tuberculosis, atherosclerosis, muscle degeneration, multiple sclerosis, cachexia, bone resorption, psoriatic arthritis, Reiter's syndrome, rheumatoid arthritis, gout, traumatic arthritis, rubella arthritis and acute synovitis. Recent evidence also links IL-1 activity to diabetes, pancreatic β cell diseases and Alzheimer's disease.

Use of a CSAID for the treatment of CSBP mediated disease states, can include, but not be limited to neurodegenerative diseases, such as Alzheimer's disease (as noted above), Parkinson's disease and multiple sclerosis, etc.

In a further aspect, this invention relates to a method of inhibiting the production of TNF in a mammal in need thereof which comprises administering to said mammal an effective amount of a compound of Formula (I) or a pharmaceutically acceptable salt thereof.

Excessive or unregulated TNF production has been implicated in mediating or exacerbating a number of diseases including rheumatoid arthritis, rheumatoid spondylitis, osteoarthritis, gouty arthritis and other arthritic conditions, sepsis, septic shock, endotoxic shock, gram negative sepsis, toxic shock syndrome, adult respiratory distress syndrome, chronic pulmonary inflammatory disease and chronic obstructive pulmonary disease, silicosis, pulmonary sarcoisosis, bone resorption diseases, such as osteoporosis, cardiac, brain and renal reperfusion injury, graft vs. host reaction, allograft rejections, fever and myalgias due to infection, such as influenza, brain infections including encephalitis (including HIV-induced forms), cerebral malaria, meningitis, ischemic and hemorrhagic stroke, cachexia secondary to infection or malignancy, cachexia secondary to acquired immune deficiency syndrome (AIDS), AIDS, ARC (AIDS related complex), keloid formation, scar tissue formation, inflammatory bowel disease, Crohn's disease, ulcerative colitis and pyresis.

Compounds of Formula (I) are also useful in the treatment of viral infections, where such viruses are sensitive to upregulation by TNF or will elicit TNF production in vivo. The viruses contemplated for treatment herein are those that produce TNF as a result of infection, or those which are sensitive to inhibition, such as by decreased replication, directly or indirectly, by the TNF inhibiting-compounds of Formula (I). Such viruses include, but are not limited to HIV-1, HIV-2 and HIV-3, Cytomegalovirus (CMV), Influenza, adenovirus and the Herpes group of viruses, such as but not limited to, Herpes Zoster and Herpes Simplex. Accordingly, in a further aspect, this invention relates to a method of treating a mammal afflicted with a human immunodeficiency virus (HIV) which comprises administering to such mammal an effective TNF inhibiting amount of a compound of Formula (I) or a pharmaceutically acceptable salt thereof.

It is also recognized that both IL-6 and IL-8 are produced during rhinovirus (HRV) infections and contribute to the pathogenesis of common cold and exacerbation of asthma associated with HRV infection (Turner et al. (1998), Clin. Infec. Dis., Vol 26, p 840; Teren et al. (1997), Am J Respir Crit Care Med vol 155, p1362; Grunberg et al. (1997), Am J Respir Crit Care Med 156:609 and Zhu et al, J Clin Invest (1996), 97:421). It has also been demonstrated in vitro that infection of pulmonary epithelial cells with HRV results in production of IL-6 and IL-8 (Subauste et al., J. Clin. Invest. 1995, 96:549.) Epithelial cells represent the primary site of infection of HRV. Therefore another aspect of the present invention is a method of treatment to reduce inflammation associated with a rhinovirus infection, not necessarily a direct effect on virus itself.

Compounds of Formula (I) may also be used in association with the veterinary treatment of mammals, other than in humans, in need of inhibition of TNF production. TNF mediated diseases for treatment, therapeutically or prophylactically, in animals include disease states such as those noted above, but in particular viral infections. Examples of such viruses include, but are not limited to, lentivirus infections such as, equine infectious anaemia virus, caprine arthritis virus, visna virus, or maedi virus or retrovirus infections, such as but not limited to feline immunodeficiency virus (FIV), bovine immunodeficiency virus, or canine immunodeficiency virus or other retroviral infections.

The compounds of Formula (I) may also be used topically in the treatment or prophylaxis of topical disease states mediated by or exacerbated by excessive cytokine production, such as by IL-1 or TNF respectively, such as inflamed joints, eczema, psoriasis and other inflammatory skin conditions such as sunburn; inflammatory eye conditions including conjunctivitis; pyresis, pain and other conditions associated with inflammation. Periodontal disease has also been implemented in cytokine production, both topically and systemically. Hence use of compounds of Formula (I) to control the inflammation associated with cytokine production in such peroral diseases such as gingivitis and periodontitis is another aspect of the present invention.

Compounds of Formula (I) have also been shown to inhibit the production of IL-8 (Interleukin-8, NAP). Accordingly, in a further aspect, this invention relates to a method of inhibiting the production of IL-8 in a mammal in need thereof which comprises administering to said mammal an effective amount of a compound of Formula (I) or a pharmaceutically acceptable salt thereof.

There are many disease states in which excessive or unregulated IL-8 production is implicated in exacerbating and/or causing the disease. These diseases are characterized by massive neutrophil infiltration such as, psoriasis, inflammatory bowel disease, asthma, cardiac, brain and renal reperfusion injury, adult respiratory distress syndrome, thrombosis and glomerulonephritis. All of these diseases are associated with increased IL-8 production which is responsible for the chemotaxis of neutrophils into the inflammatory site. In contrast to other inflammatory cytokines (IL-1, TNF, and IL-6), IL-8 has the unique property of promoting neutrophil chemotaxis and activation. Therefore, the inhibition of IL-8 production would lead to a direct reduction in the neutrophil infiltration.

The compounds of Formula (I) are administered in an amount sufficient to inhibit cytokine, in particular IL-1, IL-6, IL-8 or TNF, production such that it is regulated down to normal levels, or in some case to subnormal levels, so as to ameliorate or prevent the disease state. Abnormal levels of IL-1, IL-6, IL-8 or TNF, for instance in the context of the present invention, constitute: (i) levels of free (not cell bound) IL-1, IL-6, IL-8 or TNF greater than or equal to 1 picogram per ml; (ii) any cell associated IL-1, IL-6, IL-8 or TNF; or (iii) the presence of IL-1, IL-6, IL-8 or TNF mRNA above basal levels in cells or tissues in which IL-1, IL-6, IL-8 or TNF, respectively, is produced.

The discovery that the compounds of Formula (I) are inhibitors of cytokines, specifically IL-1, IL-6, IL-8 and TNF is based upon the effects of the compounds of Formulas (I) on the production of the IL-1, IL-8 and TNF in in vitro assays which are described herein.

As used herein, the term “inhibiting the production of IL-1 (IL-6, IL-8 or TNF)” refers to:
a) a decrease of excessive in vivo levels of the cytokine (IL-1, IL-6, IL-8 or TNF) in a human to normal or sub-normal levels by inhibition of the in release of the cytokine by all cells, including but not limited to monocytes or macrophages;
b) a down regulation, at the genomic level, of excessive in vivo levels of the cytokine (IL-1, IL-6, IL-8 or TNF) in a human to normal or sub-normal levels;
c) a down regulation, by inhibition of the direct synthesis of the cytokine (IL-1, IL-6, IL-8 or TNF) as a postranslational event; or
d) a down regulation, at the translational level, of excessive in vivo levels of the cytokine (IL, IL-6, IL-8 or TNF) in a human to normal or sub-normal levels.

As used herein, the term “TNF mediated disease or disease state” refers to any and all disease states in which TNF plays a role, either by production of TNF itself, or by TNF causing another monokine to be released, such as but not limited to IL-1, IL-6 or IL-8. A disease state in which, for instance, IL-1 is a major component, and whose production or action, is exacerbated or secreted in response to TNF, would therefore be considered a disease stated mediated by TNF.

As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epideral keratinocytes and B-lymphocytes. Lymphokines are generally referred to as being produced by lymphocyte cells. Examples of cytokines include, but are not limited to, Interleukin-1 (IL-1), Interleukin-6 (IL-6), Interleukin-8 (IL-8), Tumor Necrosis Factor-alpha (TNF-α) and Tumor Necrosis Factor beta (TNF-β).

As used herein, the term “cytokine interfering” or “cytokine suppressive amount” refers to an effective amount of a compound of Formula (I) which will cause a decrease in the in vivo levels of the cytokine to normal or sub-normal levels, when given to a patient for the prophylaxis or treatment of a disease state which is exacerbated by, or caused by, excessive or unregulated cytokine production.

As used herein, the cytokine referred to in the phrase “inhibition of a cytokine, for use in the treatment of a HIV-infected human” is a cytokine which is implicated in (a) the initiation and/or maintenance of T cell activation and/or activated T cell-mediated HIV gene expression and/or replication and/or (b) any cytokine-mediated disease associated problem such as cachexia or muscle degeneration.

As TNF-β (also known as lymphotoxin) has close structural homology with TNF-α (also known as cachectin) and since each induces similar biologic responses and binds to the same cellular receptor, both TNF-α and TNF-β are inhibited by the compounds of the present invention and thus are herein referred to collectively as “TNF” unless specifically delineated otherwise.

A member of the MAP kinase family, alternatively termed CSBP, p38, or RK, has been identified independently by several laboratories. Activation of this novel protein kinase via dual phosphorylation has been observed in different cell systems upon stimulation by a wide spectrum of stimuli, such as physicochemical stress and treatment with lipopolysaccharide or proinflammatory cytokines such as interleukin-1 and tumor necrosis factor. The cytokine biosynthesis inhibitors, of the present invention, compounds of Formula (I) have been determined to be potent and selective inhibitors of CSBP/p38/RK kinase activity. These inhibitors are of aid in determining the signaling pathways involvement in inflammatory responses. In particular, for the first time a definitive signal transduction pathway can be prescribed to the action of lipopolysaccharide in cytokine production in macrophages. In addition to those diseases already noted, treatment of stroke, neurotrauma, cardiac and renal reperfusion injury, congestive heart failure, coronary arterial bypass grafting (CABG) surgery, chronic renal failure, angiogenesis & related processes, such as cancer, thrombosis, glomerulonephritis, diabetes and pancreatic β cells, multiple sclerosis, muscle degeneration, eczema, psoriasis, sunburn, and conjunctivitis are also included.

The CSBP inhibitors were subsequently tested in a number of animal models for anti-inflammatory activity. Model systems were chosen that were relatively insensitive to cyclooxygenase inhibitors in order to reveal the unique activities of cytokine suppressive agents. The inhibitors exhibited significant activity in many such in vivo studies. Most notable are its effectiveness in the collagen-induced arthritis model and inhibition of TNF production in the endotoxic shock model. In the latter study, the reduction in plasma level of TNF correlated with survival and protection from endotoxic shock related mortality. Also of great importance are the compounds effectiveness in inhibiting bone resorption in a rat fetal long bone organ culture system. Griswold et al., (1988) Arthritis Rheum. 31:1406-1412; Badger, et al., (1989) Circ. Shock 27, 51-61; Votta et al., (1994)in vitro. Bone 15, 533-538; Lee et al., (1993). B Ann. N.Y. Acad. Sci. 696, 149-170.

Chronic diseases which have an inappropriate angiogenic component are various ocular neovasularizations, such as diabetic retinopathy and macular degeneration. Other chronic diseases which have an excessive or increased proliferation of vasculature are tumor growth and metastasis, atherosclerosis, and certain arthritic conditions. Therefore CSBP kinase inhibitors will be of utility in the blocking of the angiogenic component of these disease states.

The term “excessive or increased proliferation of vasculature inappropriate angiogenesis” as used herein includes, but is not limited to, diseases which are characterized by hemangiomas and ocular diseases.

The term “inappropriate angiogenesis” as used herein includes, but is not limited to, diseases which are characterized by vesicle proliferation with accompanying tissue proliferation, such as occurs in cancer, metastasis, arthritis and atherosclerosis.

Accordingly, the present invention provides a method of treating, including prophylaxis, a CSBP kinase mediated disease in a mammal in need thereof, preferably a human, which comprises administering to said mammal, an effective amount of a compound of Formula (I) or a pharmaceutically acceptable salt thereof.

Accordingly, the present invention provides a method of treating, including prophylaxis, the inflammatory component of a CSBP kinase mediated disease in a mammal in need thereof, preferably a human, which comprises administering to said mammal, an effective amount of a compound of Formula (I) or a pharmaceutically acceptable salt thereof.

In order to use a compound of Formula (I) or a pharmaceutically acceptable salt thereof in therapy, it will normally be formulated into a pharmaceutical composition in accordance with standard pharmaceutical practice. This invention, therefore, also relates to a pharmaceutical composition comprising an effective, non-toxic amount of a compound of Formula (I) and a pharmaceutically acceptable carrier or diluent.

Compounds of Formula (I), pharmaceutically acceptable salts thereof and pharmaceutical compositions incorporating such may conveniently be administered by any of the routes conventionally used for drug administration, for instance, orally, topically, parentally or by inhalation. The compounds of Formula (I) may be administered in conventional dosage forms prepared by combining a compound of Formula (I) with standard pharmaceutical carriers according to conventional procedures. The compounds of Formula (I) may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable character or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg. to about 1 g. When a liquid carrier is used, the preparation will be in the form of a syrup, emulsion, soft gelatin capsule, sterile injectable liquid such as an ampule or nonaqueous liquid suspension.

Compounds of Formula (I) may be administered topically, that is by non-systemic administration. This includes the application of a compound of Formula (I) externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as stearic or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Compounds of Formula (I) may be administered parentally, that is by intravenous, intramuscular, subcutaneous intranasal, intrarectal, intravaginal or intraperitoneal administration. The subcutaneous and intramuscular forms of parenteral administration are generally preferred. Appropriate dosage forms for such administration may be prepared by conventional techniques. Compounds of Formula (I) may also be administered by inhalation, that is by intranasal and oral inhalation administration. Appropriate dosage forms for such administration, such as an aerosol formulation or a metered dose inhaler, may be prepared by conventional techniques.

For all methods of use disclosed herein for the compounds of Formula (I), the daily oral dosage regimen will preferably be from about 0.1 to about 80 mg/kg of total body weight, preferably from about 0.2 to 30 mg/kg, more preferably from about 0.5 mg to 15 mg. The daily parenteral dosage regimen about 0.1 to about 80 mg/kg of total body weight, preferably from about 0.2 to about 30 mg/kg, and more preferably from about 0.5 mg to 15 mg/kg. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a compound of Formula (I) or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound of Formula (I) or a pharmaceutically acceptable salt thereof given per day for a defined number of days, can be ascertained by those skilled in the art using conventional course of treatment determination tests.

The novel compounds of Formula (I) may also be used in association with the veterinary treatment of mammals, other than humans, in need of inhibition of CSBP/p38 or cytokine inhibition or production. In particular, CSBP/p38 mediated diseases for treatment, therapeutically or prophylactically, in animals include disease states such as those noted herein in the Methods of Treatment section, but in particular viral infections. Examples of such viruses include, but are not limited to, lentivirus infections such as, equine infectious anaemia virus, caprine arthritis virus, visna virus, or maedi virus or retrovirus infections, such as but not limited to feline immunodeficiency virus (FIV), bovine immunodeficiency virus, or canine immunodeficiency virus or other retroviral infections.

The invention will now be described by reference to the following biological examples which are merely illustrative and are not to be construed as a limitation of the scope of the present invention.

### BIOLOGICAL EXAMPLES

The cytokine-inhibiting effects of compounds of the present invention may be determined by the following in vitro assays:

Assays for Interleukin-1 (IL-1), Interleukin-8 (IL-8), and Tumour Necrosis Factor (TNF) are well known in the art, and may be found in a number of publications, and patents. Representative suitable assays for use herein are described in Adams et al., U.S. Pat. No. 5,593,992, whose disclosure is incorporated by reference in its entirety.

Interleukin-1 (IL-1)

Human peripheral blood monocytes are isolated and purified from either fresh blood preparations from volunteer donors, or from blood bank buffy coats, according to the procedure of Colotta et al, J Immunol, 132, 936 (1984). These monocytes (1×10⁶) are plated in 24-well plates at a concentration of 1-2 million/ml per well. The cells are allowed to adhere for 2 hours, after which time non-adherent cells are removed by gentle washing. Test compounds are then added to the cells for 1 h before the addition of lipopolysaccharide (50 ng/ml), and the cultures are incubated at 37° C. for an additional 24 h. At the end of this period, culture supernatants are removed and clarified of cells and all debris. Culture supernatants are then immediately assayed for IL-1 biological activity, either by the method of Simon et al., J. Immunol. Methods, 84, 85, (1985) (based on ability of IL-1 to stimulate a Interleukin 2 producing cell line (EL-4) to secrete IL-2, in concert with A23187 ionophore) or the method of Lee et al., J. ImmunoTherapy, 6 (1), 1-12 (1990) (ELISA assay).

In Vivo TNF Assay:

(1) Griswold et al., Drugs Under Exp. and Clinical Res., XIX (6), 243-248 (1993); or

(2) Boehm, et al., Journal Of Medicinal Chemistry 39, 3929-3937 (1996) whose disclosures are incorporated by reference herein in their entirety.

LPS-Induced TNFα Production in Mice and Rats

In order to evaluate in vivo inhibition of LPS-induced TNFα production in rodents, both mice and rats are injected with LPS.

Mouse Method

Male Balb/c mice from Charles River Laboratories are pretreated (30 minutes) with compound or vehicle. After the 30 min. pretreat time, the mice are given LPS (lipopolysaccharide from Esherichia coli Serotype 055-85, Sigma Chemical Co., St Louis, Mo.) 25 ug/mouse in 25 ul phosphate buffered saline (pH 7.0) intraperitoneally. Two hours later the mice are killed by CO₂ inhalation and blood samples are collected by exsanguination into heparinized blood collection tubes and stored on ice. The blood samples are centrifuged and the plasma collected and stored at −20° C. until assayed for TNFα by ELISA.

Rat Method

Male Lewis rats from Charles River Laboratories are pretreated at, various times with compound or vehicle. After a determined pretreat time, the rats are given LPS (lipopolysaccharide from Esherichia coli Serotype 055-85, Sigma Chemical Co., St Louis, Mo.) 3.0 mg/kg intraperitoneally. The rats are killed by CO₂ inhalation and heparinized whole blood is collected from each rat by cardiac puncture 90 minutes after the LPS injection. The blood samples are centrifuged and the plasma collected for analysis by ELISA for TNFα levels.

ELISA Method

TNFα levels were measured using a sandwich ELISA, as described in Olivera et al., Circ. Shock, 37, 301-306, (1992), whose disclosure is incorporated by reference in its entirety herein, using a hamster monoclonal antimurine TNFα (Genzyme, Boston, Mass.) as the capture antibody and a polyclonal rabbit antimurine TNFα (Genzyme) as the second antibody. For detection, a peroxidase-conjugated goat antirabbit antibody (Pierce, Rockford, Ill.) was added, followed by a substrate for peroxidase (1 mg/ml orthophenylenediamine with 1% urea peroxide). TNFα levels in the plasma samples from each animal were calculated from a standard curve generated with recombinant murine TNFα (Genzyme).

LPS-Stimulated Cytokine Production in Human Whole Blood

Assay: Test compound concentrations were prepared at 10× concentrations and LPS prepared at 1 ug/ml (final conc. of 50 ng/ml LPS) and added in 50 uL volumes to 1.5 mL eppendorf tubes. Heparinized human whole blood was obtained from healthy volunteers and was dispensed into eppendorf tubes containing compounds and LPS in 0.4 mL volumes and the tubes incubated at 37 C. Following a 4 hour incubation, the tubes were centrifuged at 5000 rpm for 5 minutes in a TOMY microfuge, plasma was withdrawn and frozen at −80 C.

Cytokine measurement: IL-1 and/or TNF were quantified using a standardized ELISA technology. An in-house ELISA kit was used to detect human IL-1 and TNF. Concentrations of IL-1 or TNF were determined from standard curves of the appropriate cytokine and IC50 values for test compound (concentration that inhibited 50% of LPS-stimulated cytokine production) were calculated by linear regression analysis.

CSBP/p38 Kinase Assay:

This assay measures the CSBP/p38-catalyzed transfer of ³²P from [a-³²P]ATP to threonine residue in an epidermal growth factor receptor (EGFR)-derived peptide (T669) with the following sequence: KRELVEPLTPSGEAPNQALLR (residues 661-681). (See Gallagher et al., “Regulation of Stress Induced Cytokine Production by Pyridinyl Imidazoles: Inhibition of CSBP Kinase”, BioOrganic & Medicinal Chemistry, 1997, 5, 49-64).

Reactions were carried in round bottom 96 well plate (from Corning) in a 30 ml volume. Reactions contained (in final concentration): 25 mM Hepes, pH 7.5; 8 mM MgCl₂; 0.17 mM ATP (the Km_{[ATp]} of p38 (see Lee et al., Nature 300, n72 pg. 639-746 (December 1994)); 2.5 uCi of [g-³²P]ATP; 0.2 mM sodium orthovanadate; 1 mM DTT; 0.1% BSA; 10% glycerol; 0.67 mM T669 peptide; and 2-4 nM of yeast-expressed, activated and purified p38. Reactions were initiated by the addition of [gamma-32P]Mg/ATP, and incubated for 25 min. at 37° C. Inhibitors (dissolved in DMSO) were incubated with the reaction mixture on ice for 30 minutes prior to adding the 32P-ATP. Final DMSO concentration was 0.16%. Reactions were terminated by adding 10 ul of 0.3 M phosphoric acid, and phosphorylated peptide was isolated from the reactions by capturing it on p81 phosphocellulose filters. Filters were washed with 75 mM phosphoric acids, and incorporated 32P was quantified using beta scintillation counter. Under these conditions, the specific activity of p38 was 400-450 pmol/pmol enzyme, and the activity was linear for up to 2 hours of incubation. The kinase activity values were obtained after subtracting values generated in the absence of substrate which were 10-15% of total values.

Representative compounds of Formula (I), Examples 24, 25, 31 to 34, 36 to 45, and 47 to 50, all showed positive inhibitory activity in the assay at <50 uM.

Compounds of Examples 1 to 10, 12, 13, 15, 16, 18, 20, and 21 are all are chemical intermediates, and do not display activity in this assay. However, chemical intermediates of Examples 11, 14, 17, 19, 22 and 23 did demonstrate activity at <50 uM.

Compounds of Examples 26 to 30, 35, 46, and 51 have demonstrated some inhibition at the 50 uM concentration level, but due to poor solubility an IC₅₀ has not been established. However, upon retesting of compounds in this group, Example No. 35 and 46 were retested at a higher concentration (67 uM), and found to be active. Therefore, it is expected that at higher concentrations (67 um or 100 uM) the rest of the final compounds which were found to be inactive are expected to demonstrate positive inhibitory activity.

Prostaglandin Endoperoxide synthase-2 (PGHS-2) Assay:

This assay describes a method for determining the inhibitory effects of compounds of Formula (I) on human PGHS-2 protein expression in LPS stimulated human monocytes. A suitable assay for PGHS-2 protein expression may be found in a number of publications, including U.S. Pat. No. 5,593,992 whose disclosure is incorporated herein by reference.

TNF-α in Traumatic Brain Injury Assay

This assay provides for examination of the expression of tumor necrosis factor mRNA in specific brain regions which follow experimentally induced lateral fluid-percussion traumatic brain injury XI) in rats. Since TNF-α is able to induce nerve growth factor (NGF) and stimulate the release of other cytokines from activated astrocytes, this post-traumatic alteration in gene expression of TNF-α plays an important role in both the acute and regenerative response to CNS trauma. A suitable assay may be found in WO 97/35856 whose disclosure is incorporated herein by reference.

CNS Injury Model for IL-β mRNA

This assay characterizes the regional expression of interleukin-1β (IL-1β) mRNA in specific brain regions following experimental lateral fluid-percussion traumatic brain injury (TBI) in rats. Results from these assays indicate that following TBI, the temporal expression of IL-1β mRNA is regionally stimulated in specific brain regions. These regional changes in cytokines, such as IL-1β play a role in the post-traumatic pathologic or regenerative sequelae of brain injury. A suitable assay may be found in WO 97/35856 whose disclosure is incorporated herein by reference.

Angiogenesis Assay:

Described in WO 97/32583, whose disclosure is incorporated herein by reference, is an assay for determination of inflammatory angiogenesis which may be used to show that cytokine inhibition will stop the tissue destruction of excessive or inappropriate proliferation of blood vessels.

### SYNTHETIC EXAMPLES

The invention will now be described by reference to the following examples which are merely illustrative and are not to be construed as a limitation of the scope of the present invention. All temperatures are given in degrees centigrade, all solvents are highest available purity and all reactions run under anhydrous conditions in an argon atmosphere unless otherwise indicated.

In the Examples, all temperatures are in degrees Centigrade (° C.). Mass spectra were performed upon a VG Zab mass spectrometer using fast atom bombardment or on a micromass platform electrospray ionization mass spectrometer in the positive ion mode using 95:5 CH₃CN/CH₃OH with 1% formic acid as the carrier solvent, unless otherwise indicated. ¹H-NMR (hereinafter “NMR”) spectra were recorded at 250 MHz using a Bruker AM 250 or Am 400 spectrometer. Multiplicities indicated are: s=singlet, d=doublet, t=triplet, q=quartet, m=multiplet and br indicates a broad signal. Sat. indicates a saturated solution, eq indicates the proportion of a molar equivalent of reagent relative to the principal reactant.

Flash chromatography is run over Merck Silica gel 60 (230-400 mesh).

### Example 1

### Preparation of [2-(4-fluoro-2-methyl-phenyl)-9-methyl-9H-purin-6-yl]-(2,4,6-trifluoro-phenyl)-amine

### a) 2,6-dichloro-9-methylpurine

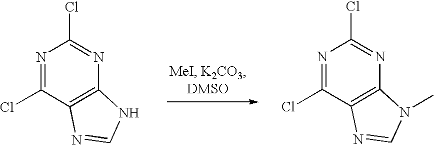

A solution of 2,6-dichloropurine (3.88 g, 20.53 mmol) in DMSO (40 mL) was treated with K₂CO₃ (3.06 g, 22.17 mmol) and MeI (1.81 mL, 29.07 mmol). The mixture was heated to 40° C. for 2 h, added to water (0° C., 100 mL), and extracted with ethyl acetate (100 mL, 3×). The combined organic phases were dried over 10 MgSO₄, filtered, and concentrated. Flash chromatography (ethyl acetate/hexane, 1:1) provided the title compound as a white solid (2.51 g, 60%).

### b) 2-chloro-9-methyl-6-methylsulfanyl-9H-purine

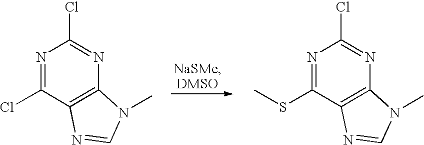

A solution of the compound of Example 1(a) (1 g, 4.93 mmol) in DMSO (30 mL) was treated with a mixture of NaSMe (417 mg, 6.0 mmol) in DMSO (34 mL) at room temperature for 2 h. The resultant mixture was then added to brine (100 mL) and extracted with EtOAc (100 mL, 3×). The combined organic phases were dried over MgSO₄, filtered, and concentrated. Flash chromatography (ethyl acetate/hexane, 4:1) afforded the title compound as a yellow solid (0.93 g, 88%). MS(ES) m/e 216 [M+H]⁺.

### c) 2-(4-fluoro-2-methyl-phenyl)-9-methyl-6-methylsulfanyl-9H-purine

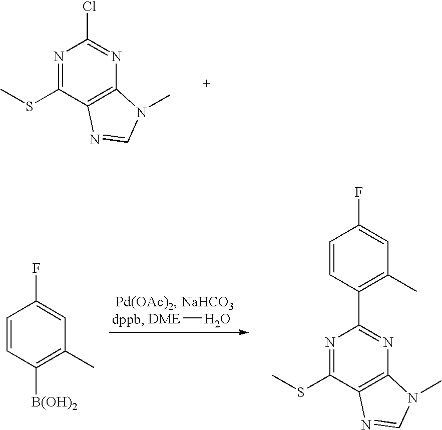

Pd(OAc)₂ (10.5 mg, 0.047 mmol) in DME (10 mL) was purged with Ar, then with stirring, under Ar, 1,4-bis(diphenylphosphino)butane (21.9 mg, 0.051 mmol) was added. The mixture was gently warmed to give an amber colored solution which was cooled to 23° C. over 0.5 h. Then in rapid succession, under Ar, was added the compound of Example 1(b) (100 mg, 0.47 mmol), 2-methyl-4-fluorophenylboronic acid (78.5 mg, 0.51 mmol), NaHCO₃ (117.6 mg, 1.4 mmol) and H₂O (0.3 mL). The resultant mixture was heated to 120° C. for 2 days in a sealed vessel. The mixture was cooled to room temperature, added to brine (50 mL), and extracted with ether (50 mL, 3×). The combined organic phases were dried over MgSO₄, filtered, and concentrated. Flash chromatography (ethyl acetate/hexane, 1:1) provided the title compound as a yellow solid (115 mg, 86%). MS(ES) m/e 289 [M+H]⁺.

### d) 2-(4-fluoro-2-methyl-phenyl)-6-methanesulfonyl-9-methyl-9H-purine

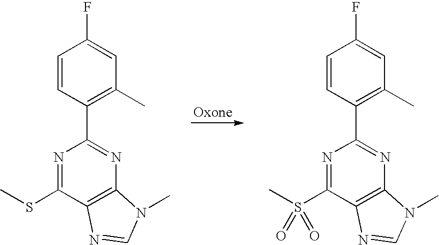

A solution of the compound of Example 1(c) (60 mg, 0.21 mmol) in THF (4 mL) was treated with a solution of Oxone (384 mg, 0.63 mmol) in H₂O (4 mL) for 21 h. The resultant mixture was added to brine (20 mL) and extracted with EtOAc (20 mL, 3×). The combined organic phases were dried over MgSO₄, filtered, and concentrated. Flash chromatography (ethyl acetate/hexane, 4:1) afforded the title compound as a white solid (52 mg, 78%). MS(ES) m/e 321 [M+H]⁺.

### e) [2-(4-fluoro-2-methyl-phenyl)-9-methyl-9H-purin-6-yl]-(2,4,6-trifluoro-phenyl)-amine

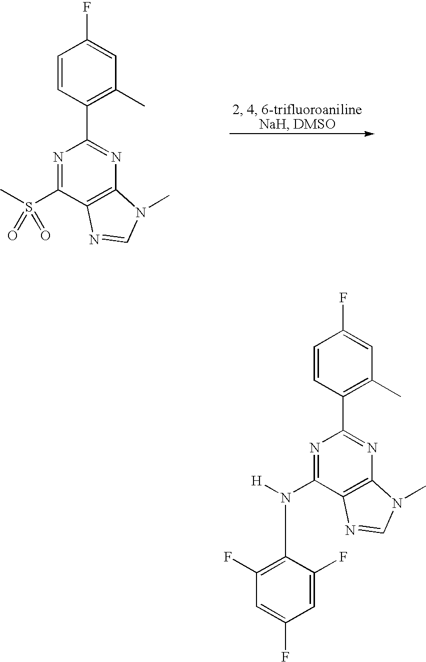

A solution of 2,4,6-trifluoroaniline (93 mg, 0.63 mmol) in DMSO (10 mL) was treated with NaH (60% in mineral oil, 25 mg, 0.63 mmol) for 20 minutes at room temperature. The resultant solution was then mixed with the compound of Example 1(d) and heated to 90° C. for 10 minutes. The mixture was added to saturated NH₄Cl solution (20 mL) and extracted with EtOAc (20 mL, 3×). The combined organic phases were dried over MgSO₄, filtered, and concentrated. Flash chromatography (ethyl acetate/hexane, 2:1) provided the title compound as a white solid (60 mg, 75%). ¹H-NMR (400 MHz, CD₃OD) δ 2.42 (s, 3H), 3.98 (s, 3H), 6.88-7.13 (m, 4H), 7.72-7.81 (m, 1H), 8.32 (s, 1H). MS(ES) m/e 388 [M+H]⁺.

### Example 2

### Preparation of (2,6-difluoro-phenol)-[2-(4-fluoro-2-methyl-phenyl)-9-methyl-9H-purin-6-yl]-amine

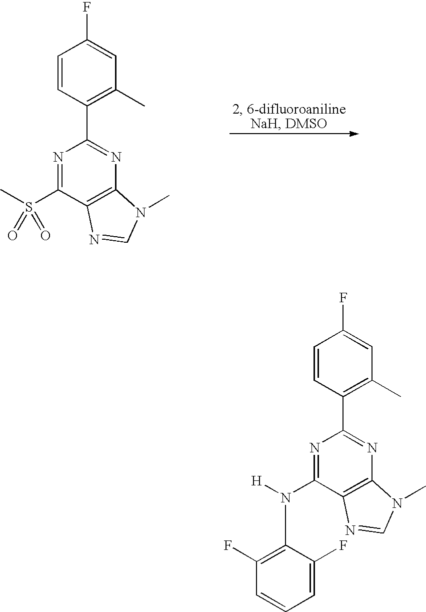

The compound of Example 1(d) (46 mg, 0.144 mmol) was reacted by the procedure of Example 1(e) except that 2,6-difluoroaniline was used instead of 2,4,6-trifluoroaniline to afford the title compound as a light yellow solid (48 mg, 90%). MS(ES) m/e 370 [M+H]⁺.

### Example 3

### Preparation of [2-(4-fluoro-2-methyl-phenyl)-9-methyl-9H-purin-6-yl]-(2-methoxy-phenyl)-amine

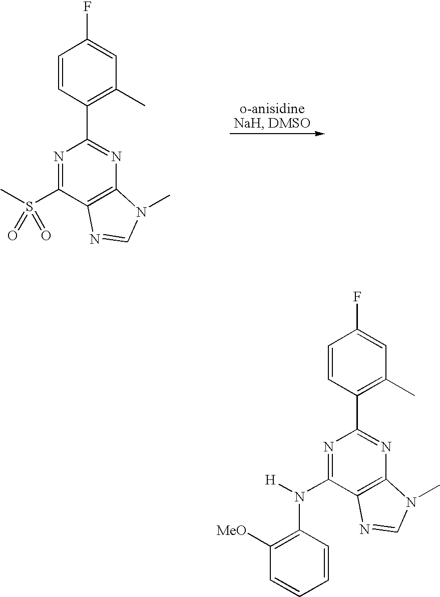

The compound of Example 1(d) (30 mg, 0.094 mmol) was reacted by the procedure of Example 1(e) except that o-anisidine was used instead of 2,4,6-trifluoroaniline to afford the title compound as a white solid (20 mg, 59%). MS(ES) m/e 364 [M+H]⁺.

### Example 4

### Preparation of [2-(4-fluoro-2-methyl-phenyl)-9-methyl-9H-purin-6-yl]-(2-fluoro-phenyl)-amine

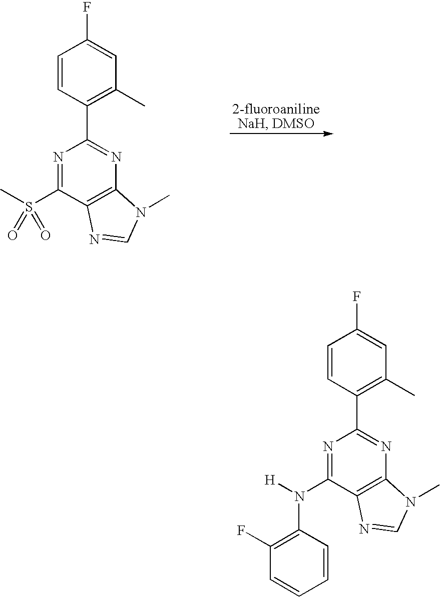

The compound of Example 1 (d) (76 mg, 0.24 mmol) was reacted by the procedure of Example 1(e) except that 2-fluoroaniline was used instead of 2,4,6-trifluoroaniline to afford the title compound as a white solid (75 mg, 90%). MS(ES) m/e 352 [M+H]⁺.

### Example 5

### Preparation of (2-chloro-phenyl)-[2-(4-fluoro-2-methyl-phenyl)-9-methyl-9H-purin-6-yl)]-amine

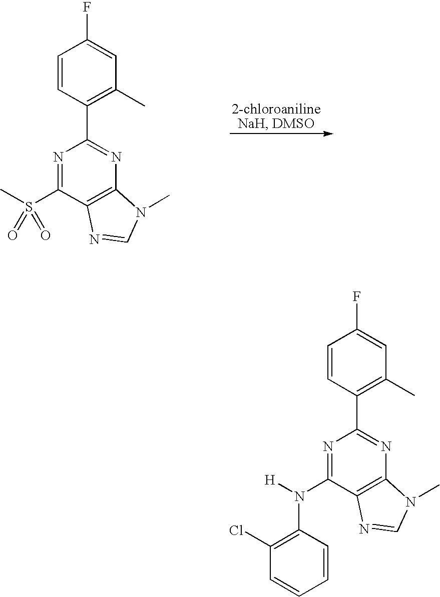

The compound of Example 1(d) (75 mg, 0.23 mmol) was reacted by the procedure of Example 1(e) except that 2-chloroaniline was used instead of 2,4,6-trifluoroaniline to afford the title compound as a white solid (74 mg, 86%). MS(ES) m/e 369 [M+H]⁺.

### Example 6

### Preparation of [2-(4-fluoro-2-methyl-phenyl)-9-methyl-9H-purin-6-yl]-(4-fluoro-phenyl)-amine

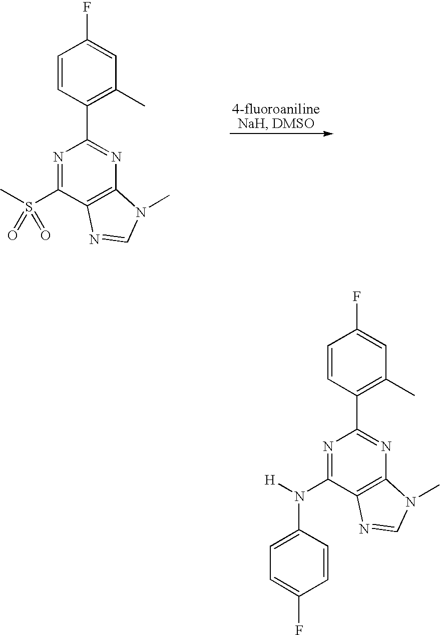

The compound of Example 1(d) (63 mg, 0.20 mmol) was reacted by the procedure of Example 1(e) except that 4-fluoroaniline was used instead of 2,4,6-trifluoroaniline to afford the title compound as a yellow solid (70 mg, 99%). MS(ES) m/e 352 [M+H]⁺.

### Example 7

### Preparation of (4-chloro-phenyl)-[2-(4-fluoro-2-methyl-phenyl)-9-methyl-9H-purin-6-yl]-amine

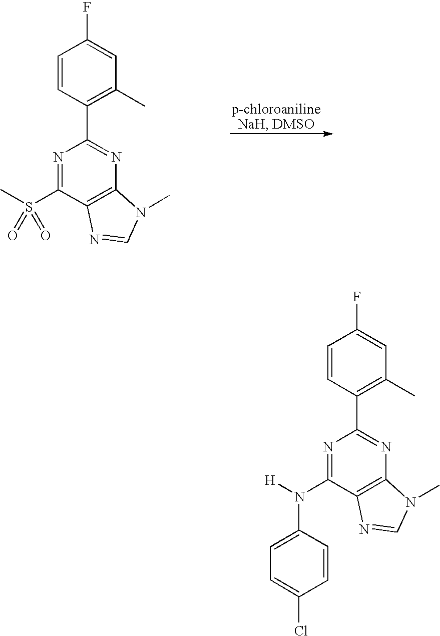

The compound of Example 1(d) (129.6 mg, 1.02 mmol) was reacted by the procedure of Example 1 (e) except that p-chloroaniline was used instead of 2,4,6-trifluoroaniline to afford the title compound as a yellow solid (52 mg, 69%). MS(ES) m/e 352 [M+H]⁺.

### Example 8

### Preparation of 2-[2-(4-fluoro-2-methyl-phenyl)-9-methyl-9H-purin-6-ylamino]-benzonitrile

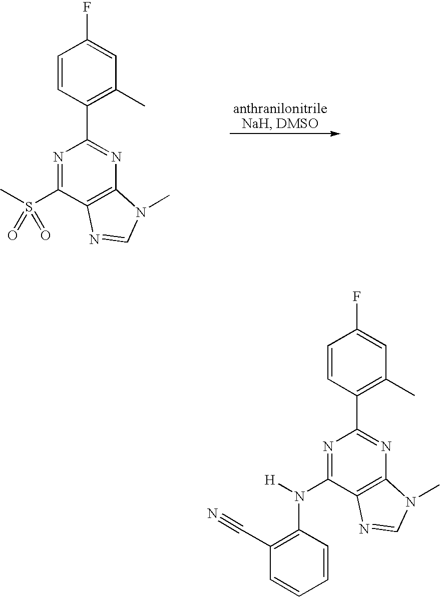

The compound of Example 1(d) (78 mg, 0.24 mmol) was reacted by the procedure of Example 1(e) except that anthranilonitrile was used instead of 2,4,6-trifluoroaniline to afford the title compound as a yellow solid (50 mg, 57%). MS(ES) m/e 359 [M+H]⁺.

### Example 9

### Preparation of [2-(4-chloro-phenyl)-9-methyl-9H-purin-6-yl]-(2,6-difluoro-phenyl)-amine

### a) (2,6-difluoro-phenyl)-(2-iodo-9-methyl-9H-purin-6-yl)-amine

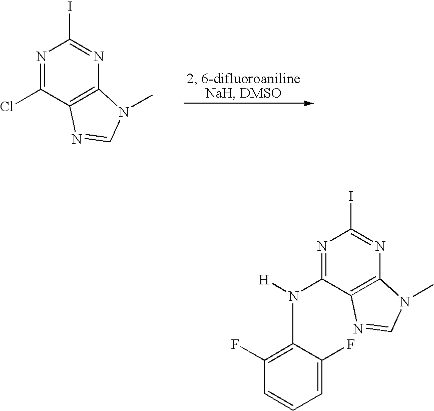

A solution of 2,6-difluoroaniline (570 mg, 4.41 mmol) in DMSO (50 mL) was treated with NaH (60% in mineral oil, 177 mg, 4.41 mmol) for 20 minutes at room temperature. The resultant solution was then mixed with 6-chloro-2-iodo-9-methylpurine (520 mg, 1.76 mmol). The mixture was heated to 80° C. for 10 minutes, added to saturated NH₄Cl (50 mL), and extracted with ether (100 mL, 3×). The combined organic phases were dried over MgSO₄, filtered, and concentrated. Flash chromatography provided the title compound as a white solid (630 mg, 92%). MS(ES) m/e 388 [M+H]⁺.

### b) [2-(4-chloro-phenyl)-9-methyl-9H-purin-6-yl]-(2,6-difluoro-phenyl)-amine

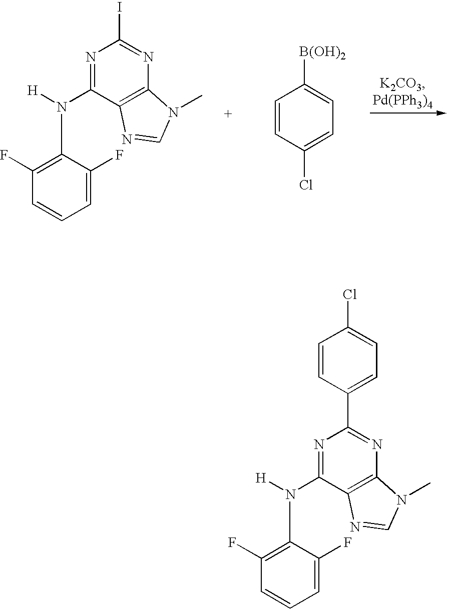

A solution of the compound of Example 9(a) (50 mg, 0.129 mmol), 4-chlorobenzeneboronic acid (30 mg, 0.194 mmol) and K₂CO₃ (54 mg, 0.387 mmol) in 1,4-dioxane/H₂O (20 mL, 3:1) was purged with Ar for 5 minutes. The resultant mixture was mixed with Pd(PPh₃)₄ and heated to 90° C. in a sealed vessel for 8 h. The mixture was cooled down to room temperature, added to brine (20 mL), and extracted with ether (30 mL, 3×). Flash chromatography (ethyl acetate/hexane, 1:1) provided the title compound as a white solid (40 mg, 79%). ¹H-NMR (400 MHz, CD₃OD) δ4.02 (s, 3H), 7.11-7.23 (m, 2H), 7.38-7.45 (m, 3H), 8.24-8.41 (m, 3H). MS(ES) m/e 373 [M+H]⁺.

### Example 10

### Preparation of [2-(3-chloro-phenyl)-9-methyl-9H-purin-6-yl]-(2,6-difluoro-phenyl)-amine

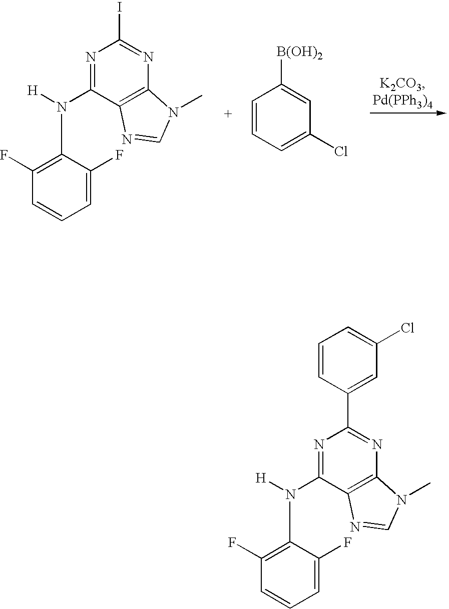

The compound of Example 9(a) (50 mg, 0.129 mmol) was reacted by the procedure of Example 9(b) except that 3-chlorobenzeneboronic acid was used instead of 4-chlorobenzeneboronic acid to afford the title compound as a white solid (41 mg, 85%). MS(ES) m/e 373 [M+H]⁺.

### Example 11

### Preparation of [2-(2-chloro-phenyl)-9-methyl-9H-purin-6-yl]-(2,6-difluoro-phenyl)-amine

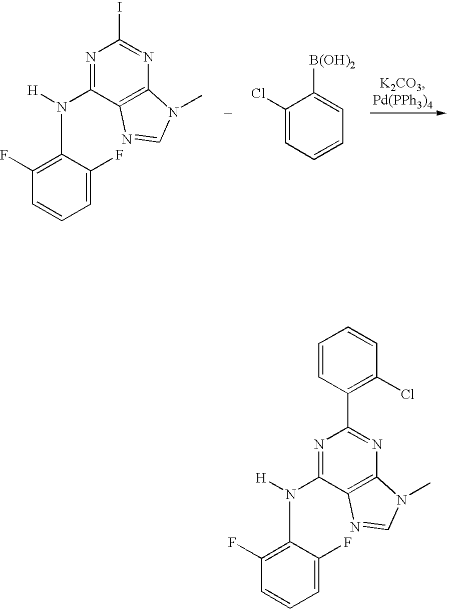

The compound of Example 9(a) (40 mg, 0.10 mmol) was reacted by the procedure of Example 9(b) except that 2-chlorobenzeneboronic acid was used instead of 4-chlorobenzeneboronic acid to afford the title compound as a white solid (35 mg, 95%). MS(ES) m/e 373 [M+H]⁺.

### Example 12

### Preparation of (2,6-difluoro-phenyl)-(9-methyl-2-phenyl-9H-purin-6-yl]-amine

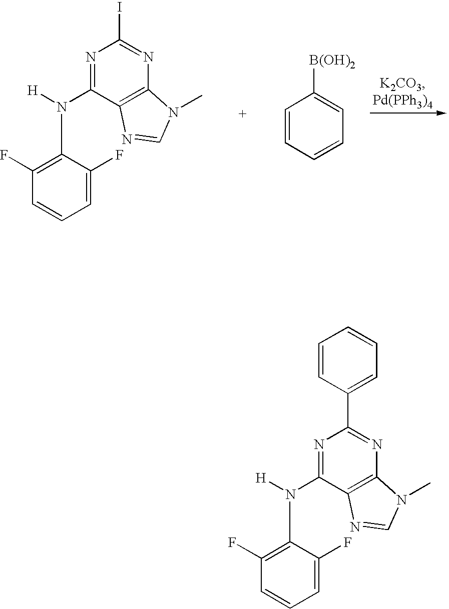

The compound of Example 9(a) (100 mg, 0.258 mmol) was reacted by the procedure of Example 9(b) except that benzeneboronic acid was used instead of 4-chlorobenzeneboronic acid to afford the title compound as a white solid (80 mg, 91%). MS(ES) m/e 338 [M+H]⁺.

### Example 13

### Preparation of (2,6-difluoro-phenyl)-(9-methyl-2-m-tolyl-9H-purin-6-yl]-amine

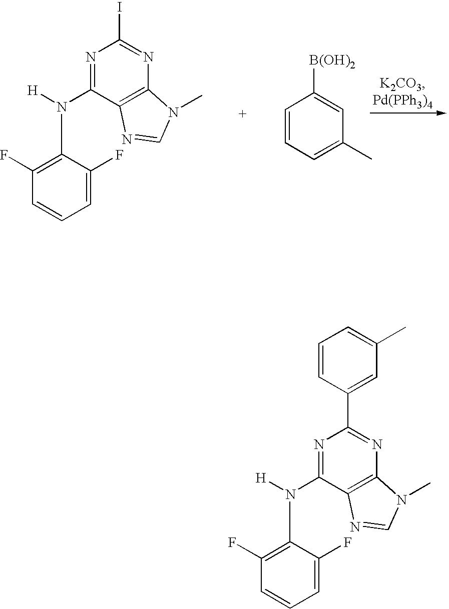

The compound of Example 9(a) (50 mg, 0.129 mmol) was reacted by the procedure of Example 9(b) except that 3-methylbenzeneboronic acid was used instead of 4-chlorobenzeneboronic acid to afford the title compound as a white solid (40 mg, 88%). MS(ES) m/e 352 [M+H]⁺.

### Example 14

### Preparation of (2,6-difluoro-phenyl)-(9-methyl-2-p-tolyl-9H-purin-6-yl)-amine

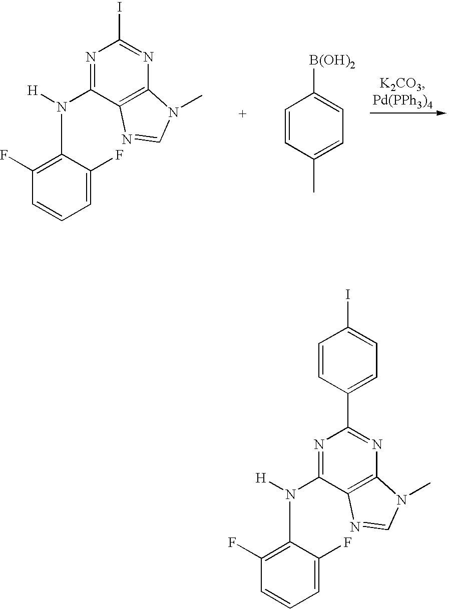

The compound of Example 9(a) (50 mg, 0.129 mmol) was reacted by the procedure of Example 9(b) except that 4-methylbenzeneboronic acid was used instead of 4-chlorobenzeneboronic acid to afford the title compound as a white solid (41 mg, 91%). MS(ES) m/e 352 [M+H]⁺.

### Example 15

### Preparation of (2,6-difluoro-phenyl)-[2-(4-fluoro-phenyl)-9-methyl-9H-purin-6-yl]-amine

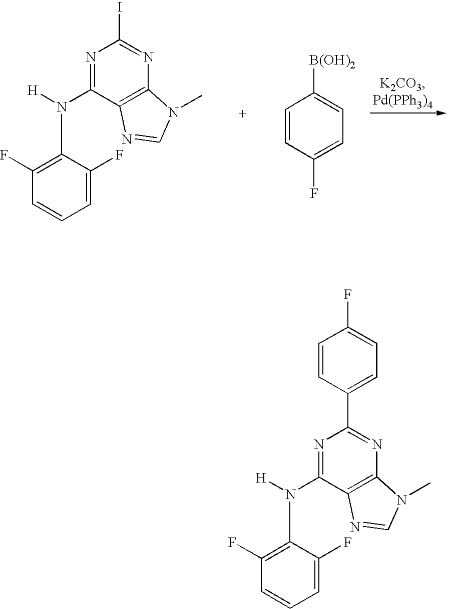

The compound of Example 9(a) (50 mg, 0.129 mmol) was reacted by the procedure of Example 9(b) except that 4-fluorobenzeneboronic acid was used instead of 4-chlorobenzeneboronic acid to afford the title compound as a white solid (42 mg, 92%). MS(ES) m/e 356 [M+H]⁺.

### Example 16

### Preparation of (2,6-difluoro-phenyl)-[2-(3-fluoro-phenyl)-9-methyl-9H-purin-6-yl]-amine

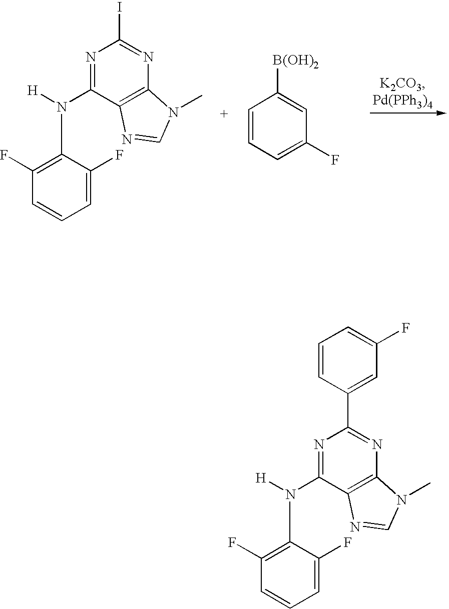

The compound of Example 9(a) (50 mg, 0.129 mmol) was reacted by the procedure of Example 9(b) except that 3-fluorobenzeneboronic acid was used instead of 4-chlorobenzeneboronic acid to afford the title compound as a white solid (35 mg, 76%). MS(ES) m/e 356 [M+H]⁺.

### Example 17

### Preparation of (2,6-difluoro-phenyl)-[2-(2-fluoro-phenyl)-9-methyl-9H-purin-6-yl]-amine

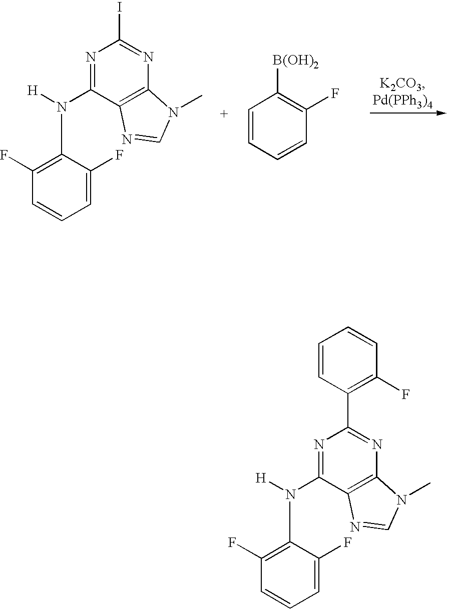

The compound of Example 9(a) (50 mg, 0.129 mmol) was reacted by the procedure of Example 9(b) except that 2-fluorobenzeneboronic acid was used instead of 4-chlorobenzeneboronic acid to afford the title compound as a white solid (46 mg, 99%). MS(ES) m/e 356 [M+H]⁺.

### Example 18

### Preparation of (2,6-difluoro-phenyl)-(9-methyl-2-o-tolyl-9H-purin-6-yl]-amine

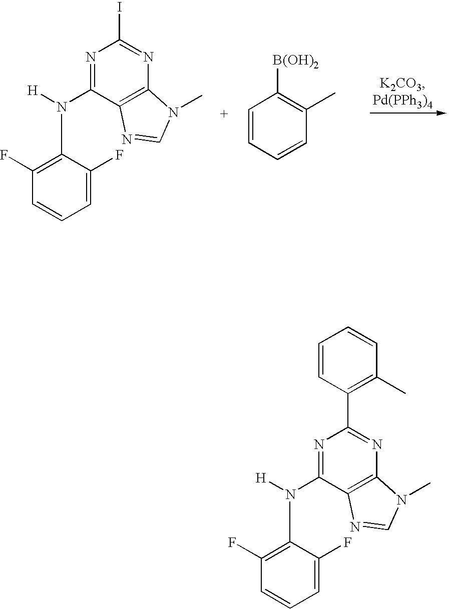

The compound of Example 9(a) (50 mg, 0.129 mmol) was reacted by the procedure of Example 9(b) except that o-tolylboronic acid was used instead of 4-chlorobenzeneboronic acid to afford the title compound as a white solid (40 mg, 88%). MS(ES) m/e 352 [M+H]⁺.

### Example 19

### Preparation of (2,6-difluoro-phenyl)-[2-(2,4-difluoro-phenyl)-9-methyl-9H-purin-6 yl]-amine

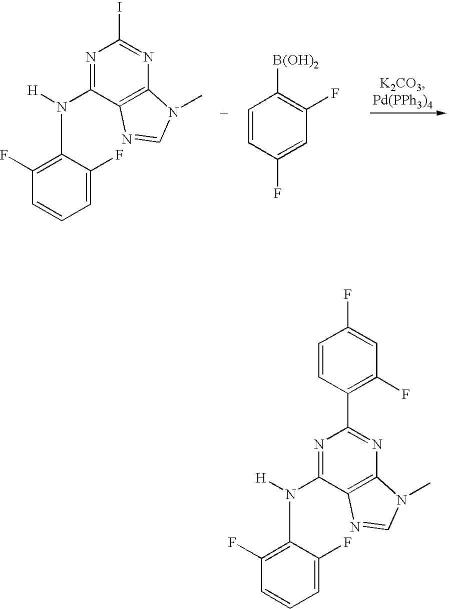

The compound of Example 9(a) (75 mg, 0.194 mmol) was reacted by the procedure of Example 9(b) except that 2,4-difluorophenylboronic acid was used instead of 4-chlorobenzeneboronic acid to afford the title compound as a white solid (72 mg, 99%). MS(ES) m/e 374 [M+H]⁺.

### Example 20

### Preparation of (2,6-difluoro-phenyl)-[2-(3,5-difluoro-phenyl)-9-methyl-9H-purin-6-yl]-amine

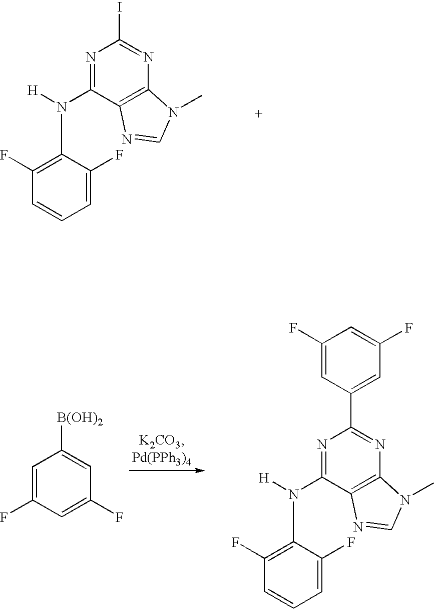

The compound of Example 9(a) (75 mg, 0.194 mmol) was reacted by the procedure of Example 9(b) except that 3,5-difluorophenylboronic acid was used instead of 4-chlorobenzeneboronic acid to afford the title compound as a white solid (73 mg, 99%). MS(ES) m/e 374 [M+H]⁺.

### Example 21

### Preparation of (2,6-difluoro-phenyl)-[2-(2-fluoro-phenyl)-9-(2-morpholin-4-yl-ethyl)-9H-purin-6-yl]-amine

### a) 6-chloro-2-iodo-9-(2-trimethylsilanyl-ethoxymethyl)-9H-purine

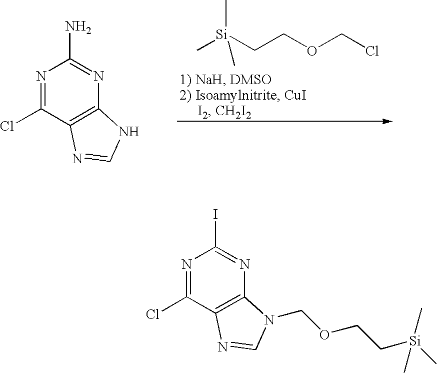

A solution of 2-amino-6-chloropurine (500 mg, 2.95 mmol) in DMSO (25 mL) was treated with NaH (60% in mineral oil, 130 mg, 3.24 mmol) and 2-(trimethylsilyl)ethoxymethyl chloride (522 μL, 2.95 mmol) for 15 h. The resultant mixture was added to brine (100 mL) and extracted with EtOAc (100 mL, 3×). The combined organic phases were dried over MgSO₄, filtered, and concentrated to provide a white solid.

The solid was mixed with THF (10 mL), 12 (186 mg, 0.73 mmol), CuI (146.6 mg, 0.77 mmol), CH₂I₂ (590 μL, 7.33 mmol) and isoamylnitrite (345 μL, 2.57 mmol). The resultant mixture was heated to 80° C. for 1 h, added to saturated NH₄Cl solution (50 mL), and extracted with ether (50 mL, 3×). The combined organic phases were dried over MgSO₄, filtered, and concentrated. Flash chromatography (ethyl acetate/hexane, 1:1) afforded the title compound as a light yellow solid (470 mg, 39% for 2 steps). MS(ES) m/e 412 [M+H]⁺.

### b) (2,6-difluoro-phenyl)-[2-iodo-9-(2-trimethylsilanyl-ethoxymethyl)-9H-purine-6-yl]-amine

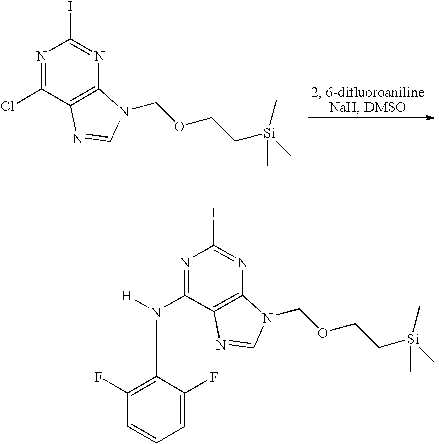

A solution of 2,6-difluoro-aniline (254 mg, 1.97 mmol) in DMSO (10 mL) was treated with NaH (60% in mineral oils, 79 mg, 1.97 mmol) at room temperature for 20 minutes. The resultant solution was mixed with the compound of Example 21(a) (270 mg, 0.657 mmol) and heated to 90° C. for 10 minutes. The mixture was added to saturated NH₄Cl solution (40 mL) and extracted with ether (50 mL, 3×). The combined organic phases were dried over MgSO₄, filtered, and concentrated. Flash chromatography (ethyl acetate/hexane, 1:4) provided the title compound as a yellow solid (280 mg, 85%). MS(ES) m/e 505 [M+H]⁺.

### c) (2,6-difluoro-phenyl)-[2-(2-fluoro-phenyl)-9-(2-trimethylsilanyl-ethoxymethyl)-9H-purine-6-yl]-amine

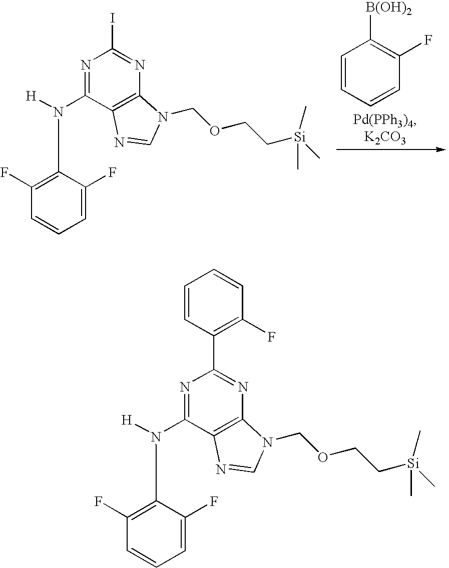

A solution of the compound of Example 21(b) (280 mg, 0.556 mmol), 2-fluorophenylboronic acid (117 mg, 0.834 mmol) and K₂CO₃ (230 mg, 1.67 mmol) in 1,4-dioxane/H₂O (3:1, 20 mL) was purged with Ar for 5 minutes. The resultant solution was mixed with Pd(PPh₃)₄ (9% Pd, 65.5 mg, 0.0556 mmol) and heated to 100° C. in a sealed vessel for 16 h. The mixture was cooled down, added to brine (40 mL), and extracted with ether (40 mL, 3×). The combined organic phases were dried over MgSO₄, filtered, and concentrated. Flash chromatography (ethyl acetate/hexane, 1:4) afforded the title compound as a white solid (236 mg, 90%). MS(ES) m/e 473 [M+H]⁺.

### d) (2,6-difluoro-phenyl)-[2-(2-fluoro-phenyl)-9H-purine-6-yl)-amine

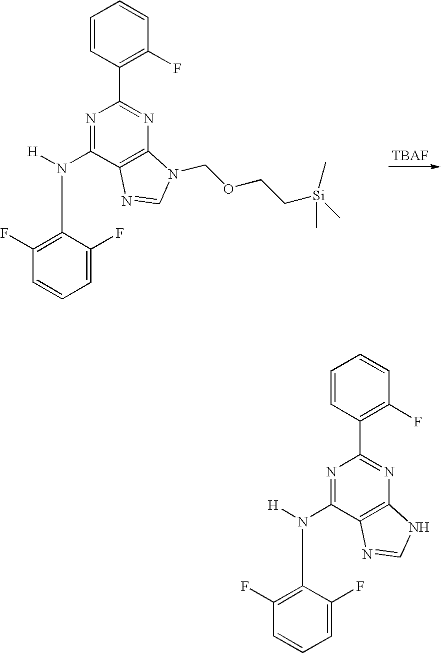

A solution of the compound of Example 21(c) (236 mg, 0.5 mmol) in THF (5 mL) was treated with TBAF (1M, 2.0 mL, 2.0 mmol) and molecular sieves (4 A° powder, 0.5 g). The mixture was heated to reflux for 1 h, added to saturated NH₄Cl solution (40 mL), and extracted with EtOAc (40 mL, 3×). The combined organic phases were dried over MgSO₄, filtered, and concentrated. Flash chromatography (ethyl acetate/hexane, 4:1) provided the title compound as a white solid (140 mg, 82%). MS(ES) m/e 342 [M+H]⁺.

### e) (2,6-difluoro-phenyl)-[2-(2-fluoro-phenyl)-9-(2-morpholin-4-yl-ethyl)-9H-purin-6-yl]-amine

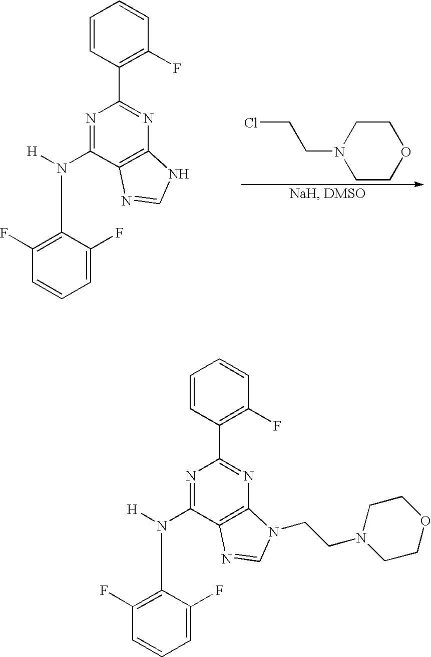

A solution of the compound of Example 21(d) (65 mg, 0.19 mmol), NaH (60% in mineral oil, 23 mg, 0.57 mmol) and 4-(2-chloroethyl)morpholine hydrochloride (53 mg, 0.29 mmol) in DMSO (10 mL) was heated to 60° C. for 17 h. The mixture was then added to brine (20 mL) and extracted with ethyl acetate (30 mL, 3×). The combined organic phases were dried over MgSO₄, filtered, and concentrated. Flash chromatography (ethyl acetate/hexane, 4:1) afforded the title compound as a yellow solid (30 mg, 35%). ¹H-NMR (400 MHz, CDCl₃) δ 2.51 (t, J=4.6 Hz, 4H), 2.92 (t, J=6 Hz, 2H), 3.87 (t, J=4.6 Hz, 4H), 4.45 (t, J=6 Hz, 2H), 7.02-7.53 (m, 8H), 8.06-8.12 (m, 1H). MS(ES) m/e 455 [M+H]⁺.

### Example 22

### Preparation of (2,6-difluoro-phenyl)-[9-(2-dimethylamino-ethyl)-2-(2-fluoro-phenyl)-9H-purin-6-yl]-amine

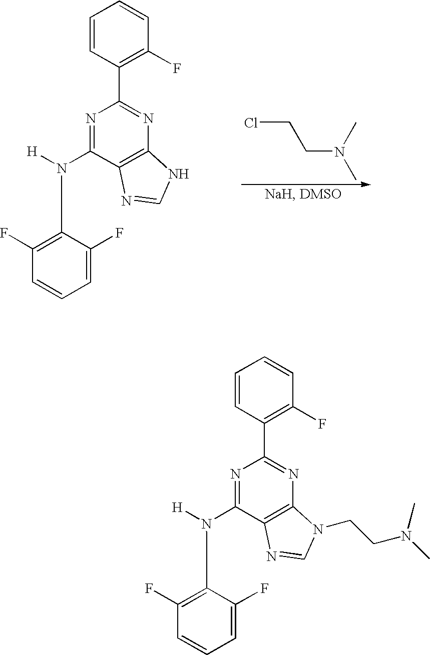

The compound of Example 21(d) (60 mg, 0.176 mmol) was reacted by the procedure of Example 21(e) except that 2-dimethylaminoethyl chloride hydrochloride was used instead of 4-(2-chloroethyl)morpholine hydrochloride to afford the title compound as a white solid (25 mg, 34%). MS(ES) m/e 414 [M+H]⁺.

### Example 23

### Preparation of [9-benzyl-2-(2-fluoro-phenyl)-9H-purin-6-yl]-(2,6-difluoro-phenyl-amine

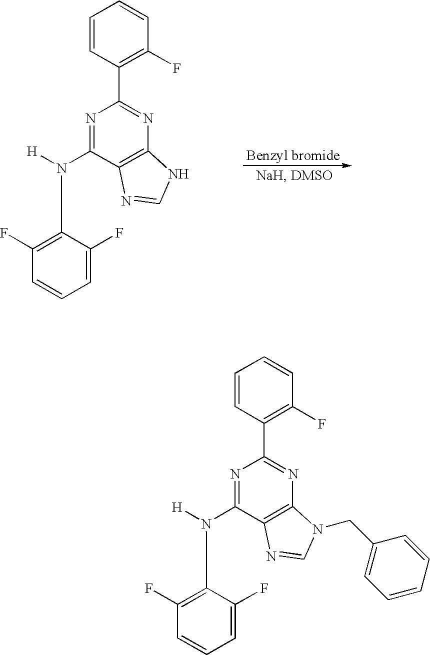

The compound of Example 21(d) (87 mg, 0.255 mmol) was reacted by the procedure of Example 21(e) except that benzyl bromide was used instead of 4-(2-chloroethyl)morpholine hydrochloride to afford the title compound as a white solid (40 mg, 36%). MS(ES) m/e 432 [M+H]⁺.

### Example 24

### Preparation of 1-[2-(4-fluoro-2-methyl-phenyl)-9-methyl-9H-purin-6-yl]-1-(2,4,6-trifluoro-phenyl)-urea

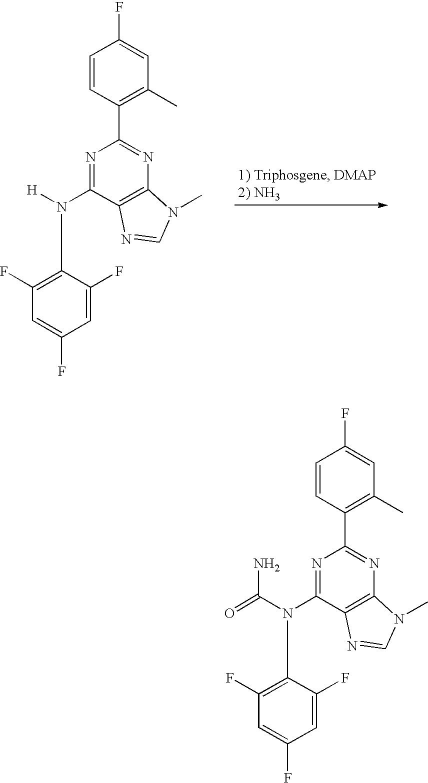

A solution of the compound of Example 1(e) (25 mg, 0.065 mmol) in THF (10 mL) was mixed with DMAP (0.8 mg, 0.0065 mmol), triethyl amine (180 μL, 1.3 mmol) and triphosgene (96 mg, 0.325 mmol) for 2 h. The resultant mixture was then treated with ammonia (38%, 2 mL) for 5 minutes. The solution was added to brine (30 mL) and extracted with EtOAc (30 mL, 3×). The combined organic phases were dried over MgSO₄, filtered, and concentrated. Purification with reverse phase HPLC afforded the title compound as a white solid (10 mg, 36%). ¹H-NMR (400 MHz, d₆-DMSO) δ 4.02 (s, 3H), 4.40 (br, 2H), 7.06-8.08 (m, 5H), 8.40 (s, 1H). MS(ES) m/e 431 [M+H]⁺.

### Example 25

### Preparation of 1-(2,6-difluoro-phenyl)-1-[2-(4-fluoro-2-methyl-phenyl)-9-methyl-9H-purin-6-yl]-urea

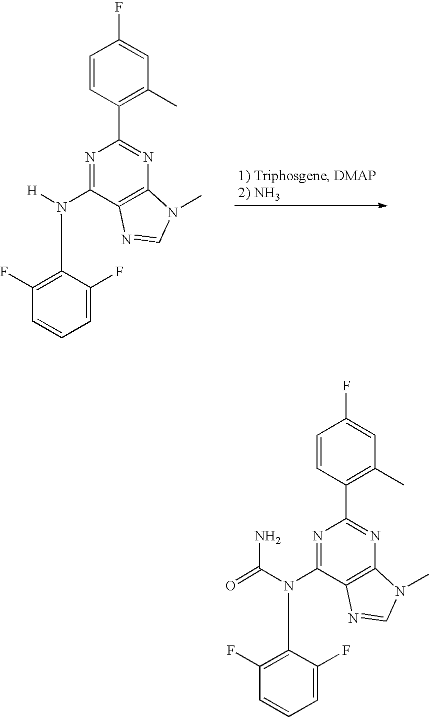

The compound of Example 2 was reacted by the procedure of Example 24 to afford the title compound as a light yellow solid. MS(ES) m/e 413 [M+H]⁺.

### Example 26

### Preparation of 1-benzyl-1-[2-(4-fluoro-2-methyl-phenyl)-9-methyl-9H-purin-6-yl]-urea

### a) benzyl-[2-(4-fluoro-2-methyl-phenyl)-9-methyl-9H-purin-6-yl]-amine

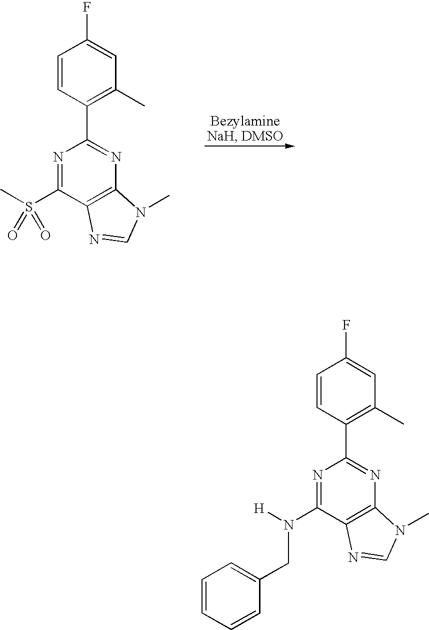

The compound of Example 1(d) (20 mg, 0.062 mmol) was reacted by the procedure of Example 1(e) except that benzyl amine was used instead of 2,4,6-trifluoroaniline to afford the title compound as a light yellow solid (20 mg, 92%). MS(ES) m/e 348 [M+H]⁺.

### b) 1-benzyl-1-[2-(4-fluoro-2-methyl-phenyl)-9-methyl-9H-purin-6-yl]-urea

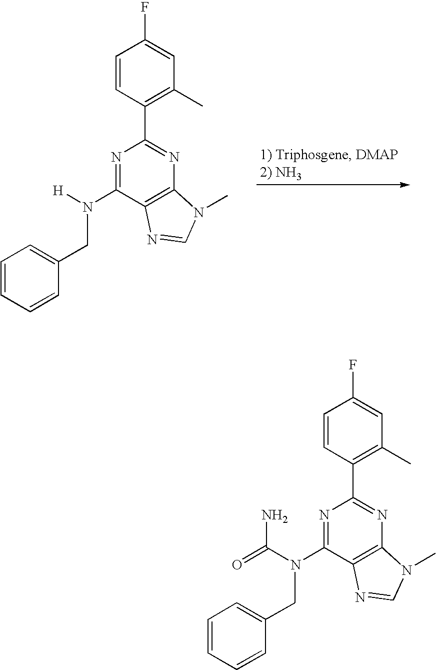

The compound of Example 26(a) was reacted by the procedure of Example 24 to afford the title compound as a white solid. MS(ES) m/e 391 [M+H]⁺.

### Example 27

### Preparation of 1-[2-(4-fluoro-2-methyl-phenyl)-9-methyl-9H-purin-6-yl]-1-[(s)-1-phenyl-ethyl]-urea

### a) [2-(4-fluoro-2-methyl-phenyl)-9-methyl-9H-purin-6-yl]-[(s)-1-phenyl-ethyl]-amine

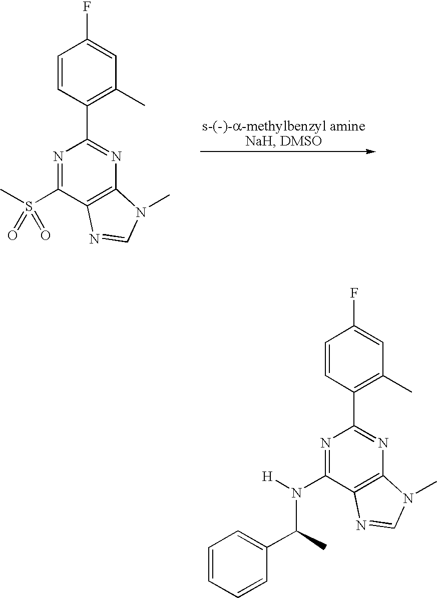

The compound of Example 1(d) (30 mg, 0.094 mmol) was reacted by the procedure of Example 1(e) except that s-(−)-α-methylbenzyl amine was used instead of 2,4,6-trifluoroaniline to afford the title compound as a yellow solid (18 mg, 53%). MS(ES) m/e 362 [M+H]⁺.

### b) 1-[2-(4-fluoro-2-methyl-phenyl)-9-methyl-9H-purin-6-yl]-1-[(s)-1-phenyl-ethyl]-urea

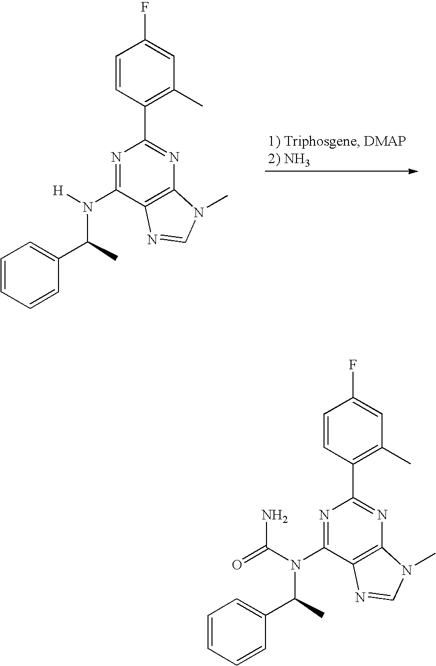

The compound of Example 27(a) was reacted by the procedure of Example 24 to afford the title compound as a light yellow solid. MS(ES) m/e 405 [M+H]⁺.

### Example 28

### Preparation of 1-[2-(4-fluoro-2-methyl-phenyl)-9-methyl-9H-purin-6-yl]-1-phenyl-urea

### a) [2-(4-fluoro-2-methyl-phenyl)-9-methyl-9H-purin-6-yl]-phenyl-amine

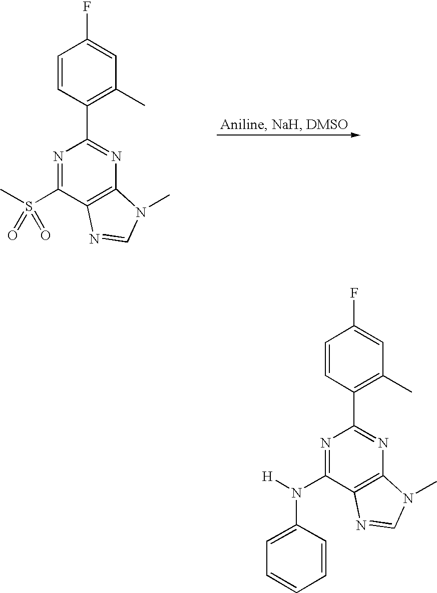

The compound of Example 1(d) (46 mg, 0.144 mmol) was reacted by the procedure of Example 1(e) except that aniline was used instead of 2,4,6-trifluoroaniline to afford the title compound as a light yellow oil (23 mg, 48%). MS(ES) m/e 334 [M+H]⁺.

### b) 1-[2-(4-fluoro-2-ethyl-phenyl)-urea

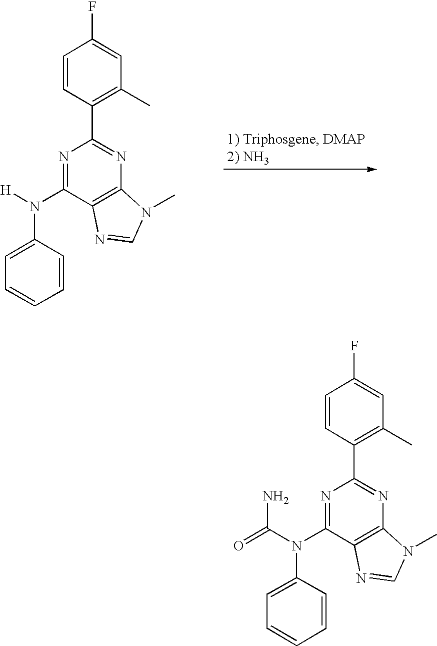

The compound of Example 28(a) was reacted by the procedure of Example 24 to afford the title compound as a yellow solid. MS (ES) m/e 377 [M+H)⁺.

### Example 29

### Preparation of 1-[2-(4-fluoro-2-methyl-phenyl)-9-methyl-9H-purin-6-yl]-(2-methoxy-phenyl)-urea

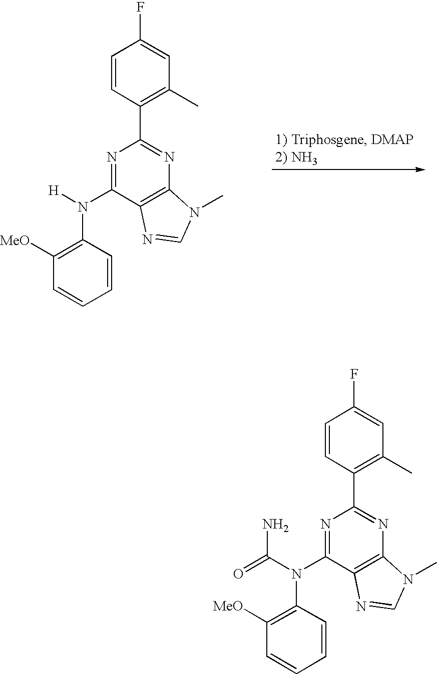

The compound of Example 3 was reacted by the procedure of Example 24 to afford the title compound as a yellow solid. MS(ES) m/e 407 [M+H]⁺.

### Example 30

### Preparation of 1-[2-(4-fluoro-2-methyl-phenyl)-9-methyl-9H-purin-6-yl]-1-(2-fluoro-phenyl)-urea

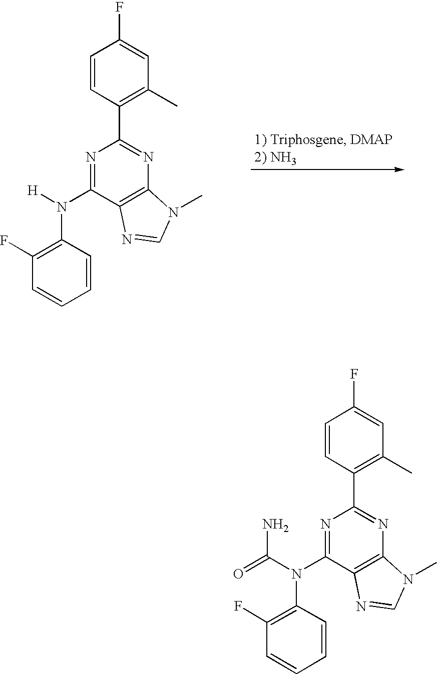

The compound of Example 4 was reacted by the procedure of Example 24 to afford the title compound as a white solid. MS(ES) m/e 395 [M+H]⁺.

### Example 31

### Preparation of 1-(2-chloro-phenyl)-1-[2-(4-fluoro-2-methyl-phenyl)-9-methyl-9H-purin-6-yl)]-urea

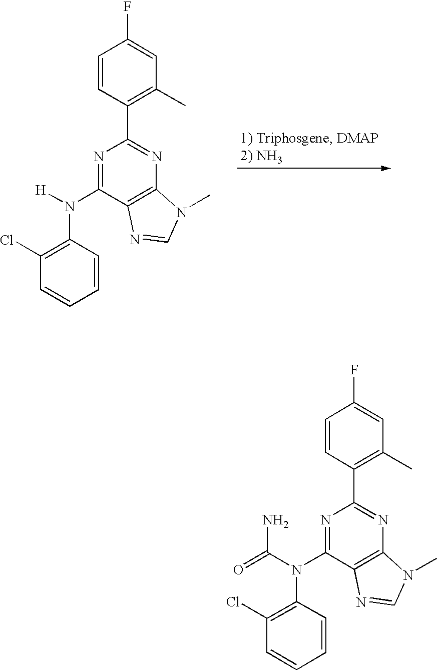

The compound of Example 5 was reacted by the procedure of Example 24 to afford the title compound as a white solid. MS(ES) m/e 412 [M+H]⁺.

### Example 32

### Preparation of 1-[2-(4-fluoro-2-methyl-phenyl)-9-methyl-9H-purin-6-yl]-1-(4-fluoro-phenyl)-urea

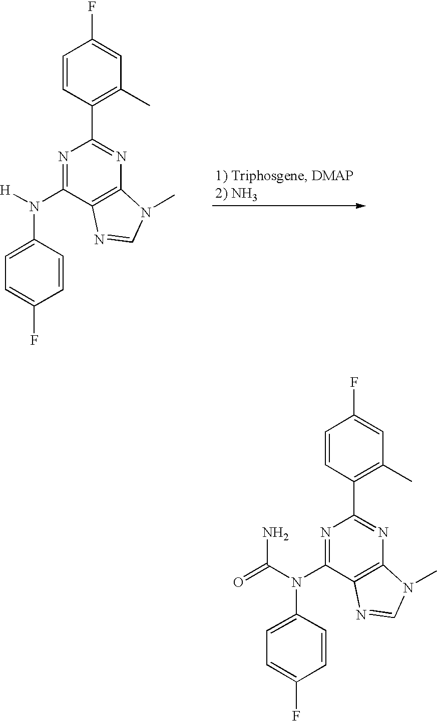

The compound of Example 6 was reacted by the procedure of Example 24 to afford the title compound as a white solid. MS(ES) m/e 395 [M+H]⁺.

### Example 33

### Preparation of 1-(4-chloro-phenyl)-1-[2-(4-fluoro-2-methyl-phenyl)-9-methyl-9H-purin-6-yl]-urea

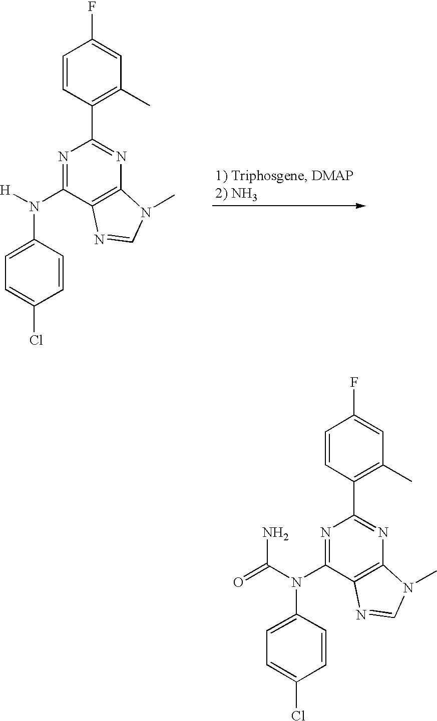

The compound of Example 7 was reacted by the procedure of Example 24 to afford the title compound as a white solid. MS(ES) m/e 411 [M+H]⁺.

### Example 34

### Preparation of 1-(4-fluoro-benzyl)-1-[2-(4-fluoro-2-methyl-phenyl)-9-methyl-9H-purin-6-yl]-urea

### a) (4-fluoro-benzyl)-[2-(4-fluoro-2-methyl-phenyl)-9-methyl-9H-purin-6-yl]-amine

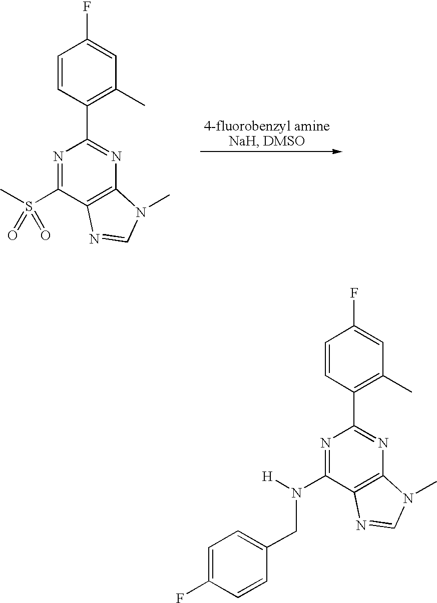

The compound of Example 1(d) (153 mg, 0.24 mmol) was reacted by the procedure of Example 1(e) except that 4-fluorobenzylamine was used instead of 2,4,6-trifluoroaniline to afford the title compound as a white solid (28 mg, 6.3%). MS(ES) m/e 366 [M+H]⁺.

### b) 1-(4-fluoro-benzyl)-1-[2-(4-fluoro-2-methyl-phenyl)-9-methyl-9H-purin-6-yl]-urea

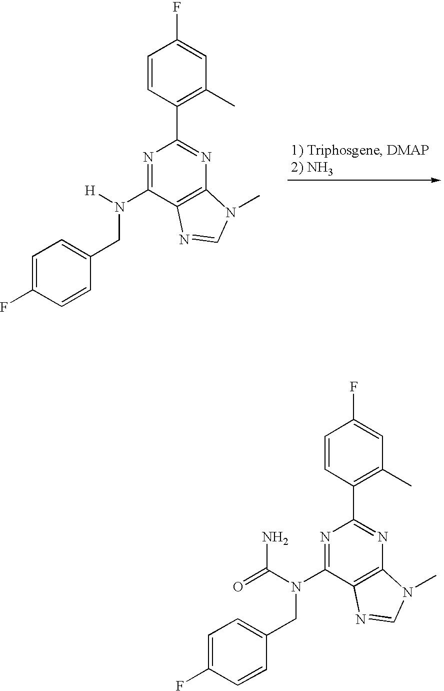

The compound of Example 34(a) was reacted by the procedure of Example 24 to afford the title compound as a white solid. MS(ES) m/e 409 [M+H]⁺.

### Example 35

### Preparation of 1-[2-(4-chloro-phenyl)-9-methyl-9H-purin-6-yl]-1-(2,6-difluoro-phenyl)-urea

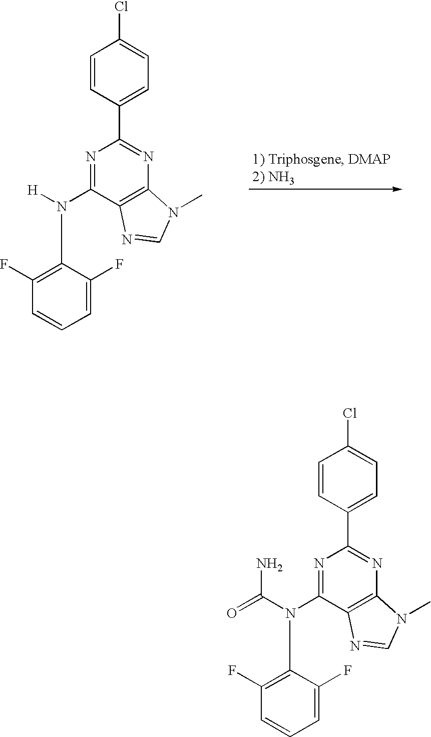

The compound of Example 9(b) was reacted by the procedure of Example 24 to afford the title compound as a white solid. MS (ES) m/e 415 [M+H]⁺.

### Example 36

### Preparation of 1-[2-(3-chloro-phenyl)-9-methyl-9H-purin-6-yl]-1-(2,6-difluoro-phenyl)-urea

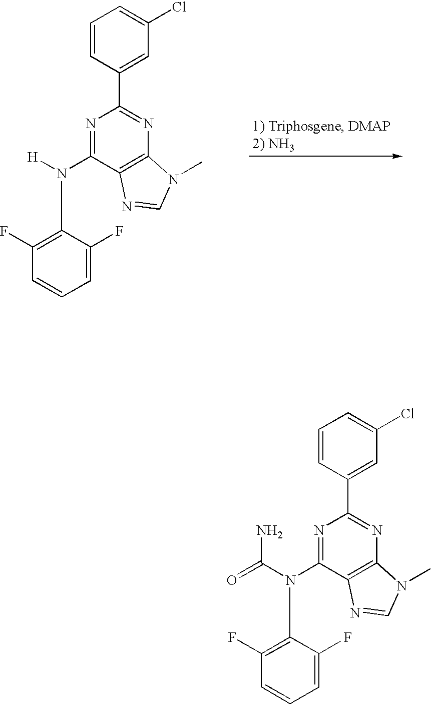

The compound of Example 10 was reacted by the procedure of Example 24 to afford the title compound as a white solid. MS(ES) m/e 415 [+H]⁺.

### Example 37

### Preparation of 1-[2-(2-chloro-phenyl)-9-methyl-9H-purin-6-yl]-1-(2,6-difluoro-phenyl)-urea

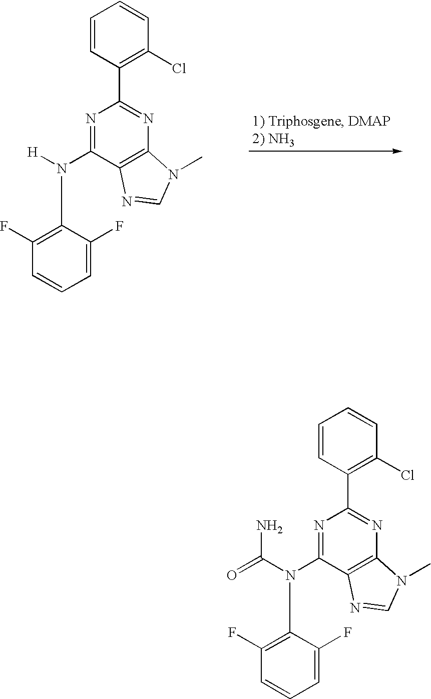

The compound of Example 11 was reacted by the procedure of Example 24 to afford the title compound as a white solid. MS(ES) m/e 415 [M+H]⁺.

### Example 38

### Preparation of 1-(2,6-difluoro-phenyl)-1-(9-methyl-2-phenyl-9H-purin-6-yl]-urea

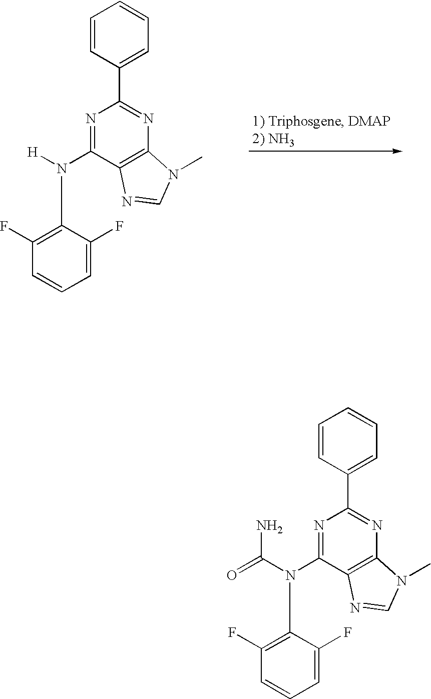

The compound of Example 12 was reacted by the procedure of Example 24 to afford the title compound as a white solid. MS(ES) m/e 381 [M+H]⁺.

### Example 39

### Preparation of 1-(2,6-difluoro-phenyl)-1-(9-methyl-2-m-tolyl-9H-purin-6-yl]-urea

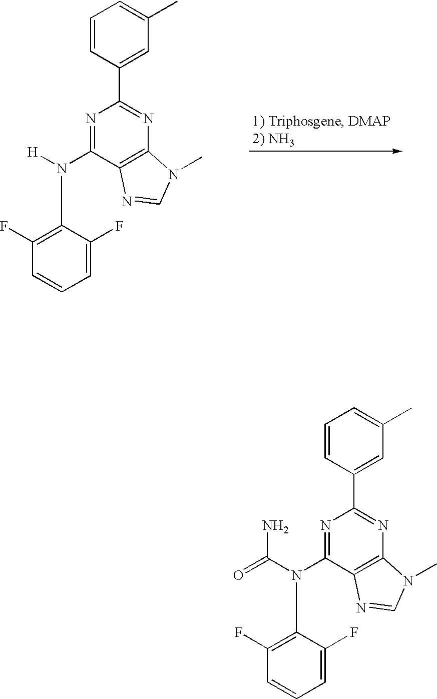

The compound of Example 13 was reacted by the procedure of Example 24 to afford the title compound as a white solid. MS(ES) m/e 395 [M+H]⁺.

### Example 40

### Preparation of 1-(2,6-difluoro-phenyl)-1-(9-methyl-2-p-tolyl-9H-purin-6-yl)-urea

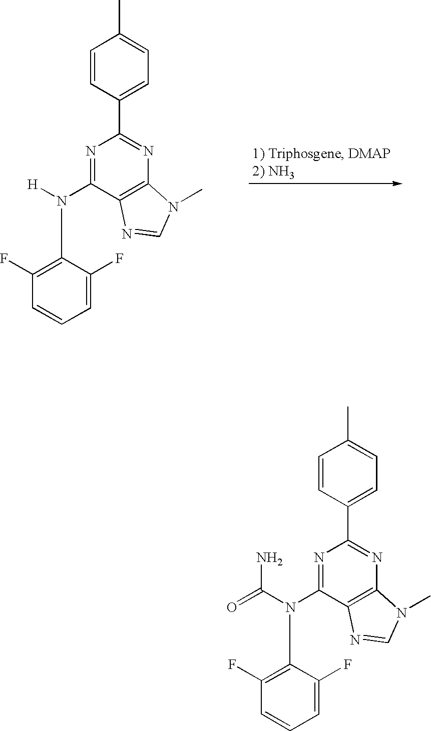

The compound of Example 14 was reacted by the procedure of Example 24 to afford the title compound as a white solid. MS(ES) m/e 395 [M+H]⁺.

### Example 41

### Preparation of 1-(2,6-difluoro-phenyl)-1-[2-(4-fluoro-phenyl)-9-methyl-9H-purin-6-yl]-urea

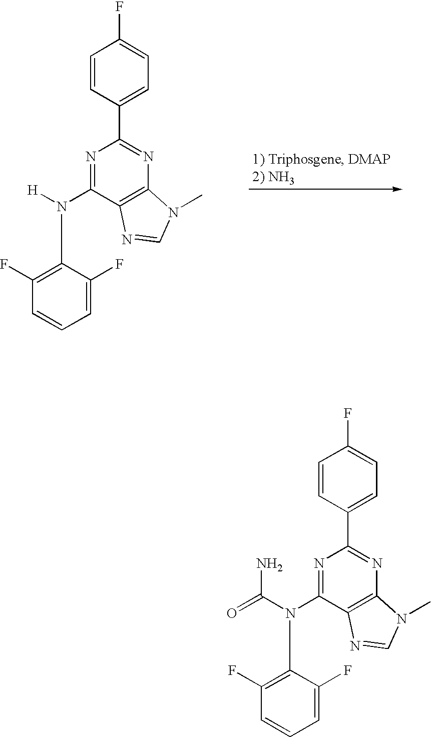

The compound of Example 15 was reacted by the procedure of Example 24 to afford the title compound as a white solid. MS(ES) m/e 399 [M+H]⁺.

### Example 42

### Preparation of 1-(2,6-difluoro-phenyl)-1-[2-(3-fluoro-phenyl)-9-methyl-9H-purin-6-yl]-urea

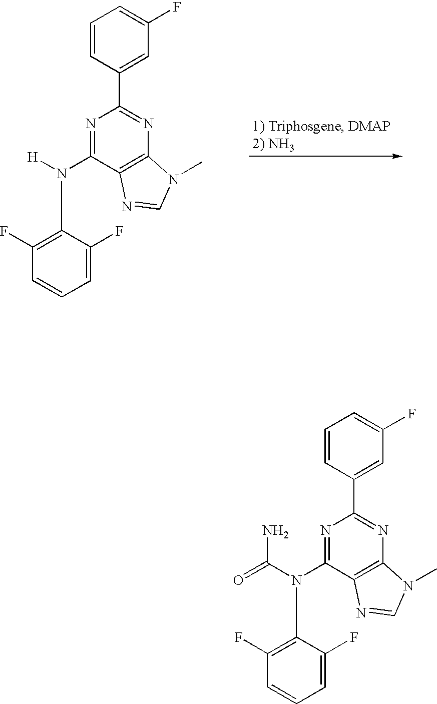

The compound of Example 16 was reacted by the procedure of Example 24 to afford the title compound as a white solid. MS(ES) m/e 399 [M+H]⁺.

### Example 43

### Preparation of 1-(2,6-difluoro-phenyl)-1-[2-(2-fluoro-phenyl)-9-methyl-9H-purin-6-yl]-urea

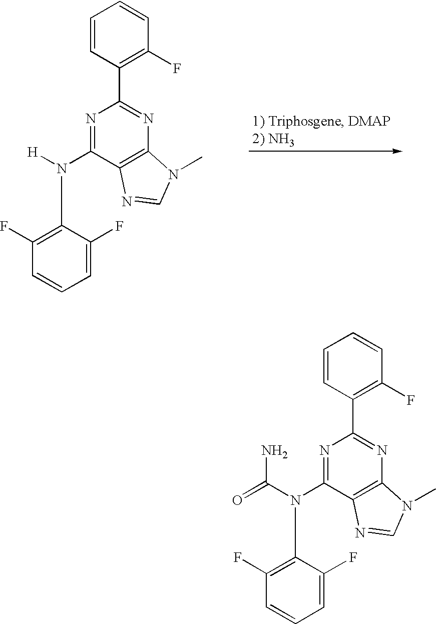

The compound of Example 17 was reacted by the procedure of Example 24 to afford the title compound as a white solid. MS(ES) m/e 399 [M+H]⁺.

### Example 44

### Preparation of 1-(2,6-difluoro-phenyl)-1-(9-methyl-2-o-tolyl-9H-purin-6-yl]-urea

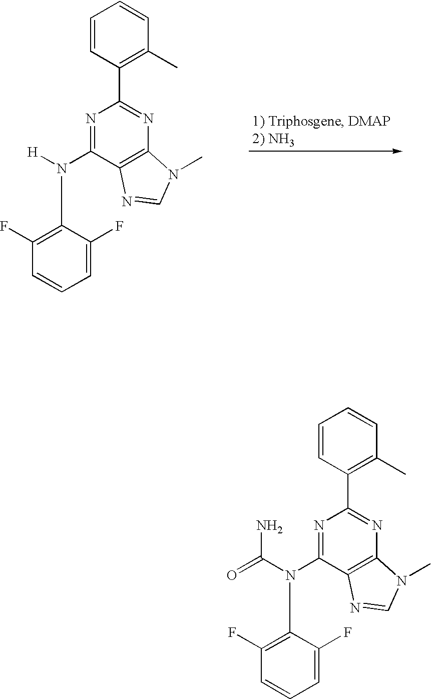

The compound of Example 18 was reacted by the procedure of Example 24 to afford the title compound as a white solid. MS(ES) m/e 395 [M+H]⁺.

### Example 45

### Preparation of 1-(2,6-difluoro-phenyl)-1-[2-(2,4-difluoro-phenyl)-9-methyl-9H-purin-6-yl]-urea

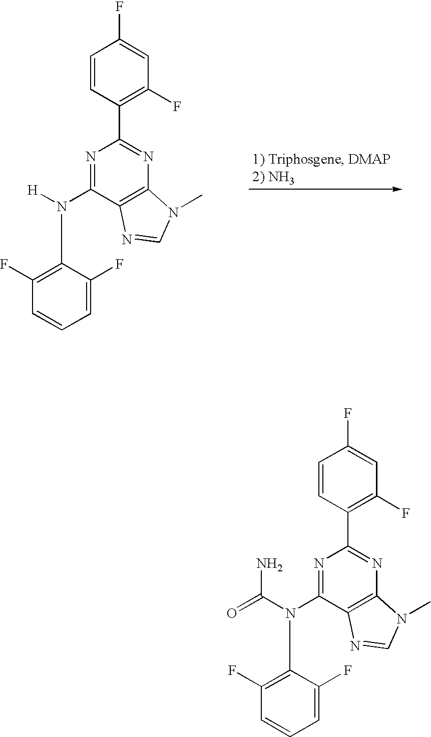

The compound of Example 19 was reacted by the procedure of Example 24 to afford the title compound as a white solid. MS(ES) m/e 417 [M+H]⁺.

### Example 46

### Preparation of 1-(2,6-difluoro-phenyl)-1-[2-(3,5-difluoro-phenyl)-9-methyl-9H-purin-6-yl]-urea

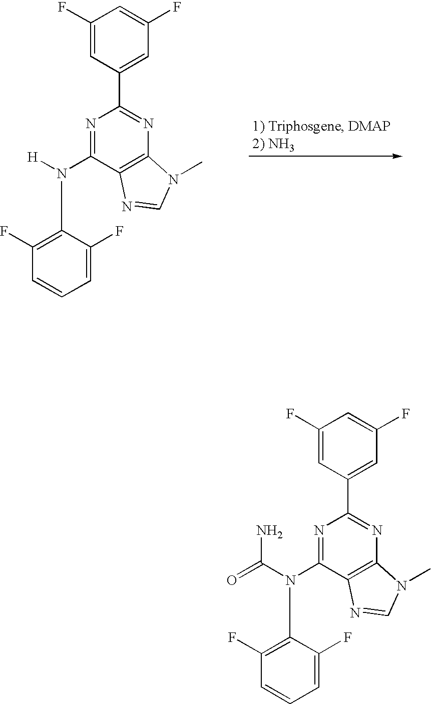

The compound of Example 20 was reacted by the procedure of Example 24 to afford the title compound as a white solid. MS(ES) m/e 417 [M+H]⁺.

### Example 47

### Preparation of 1-(2,6-difluoro-phenyl)-1-[9-(2-dimethylamino-ethyl)-2-(2-fluoro-phenyl)-9H-purin-6-yl]-urea

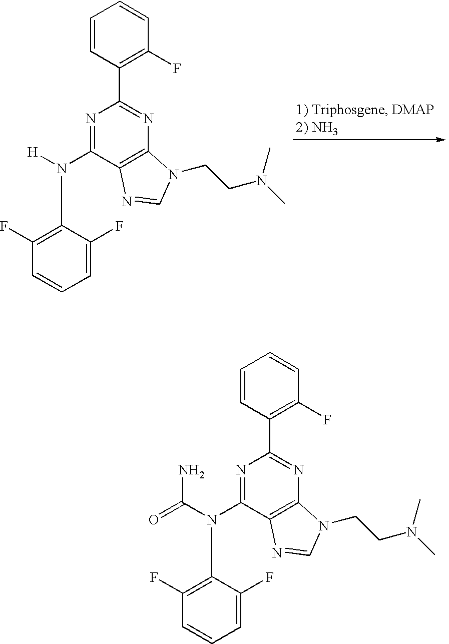

The compound of Example 22 was reacted by the procedure of Example 24 to afford the title compound as a white solid. MS(ES) m/e 456 [+H]⁺.

### Example 48

### Preparation of 1-[9-benzyl-2-(2-fluoro-phenyl)-9H-purin-6-yl]-1-(2,6-difluoro-phenyl-urea

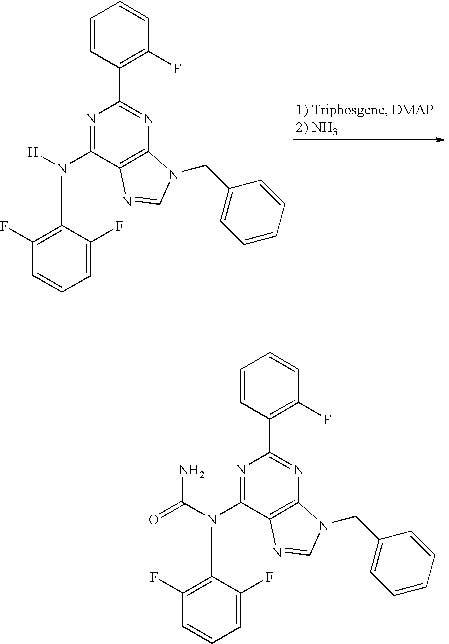

The compound of Example 23 was reacted by the procedure of Example 24 to afford the title compound as a white solid. MS(ES) m/e 475 [M+H]⁺.

### Example 49

### Preparation of 1-(2,6-difluoro-phenyl)-1-[2-(2-fluoro-phenyl)-9-(2-morpholin-4-yl-ethyl)-9H-purin-6-yl]-urea

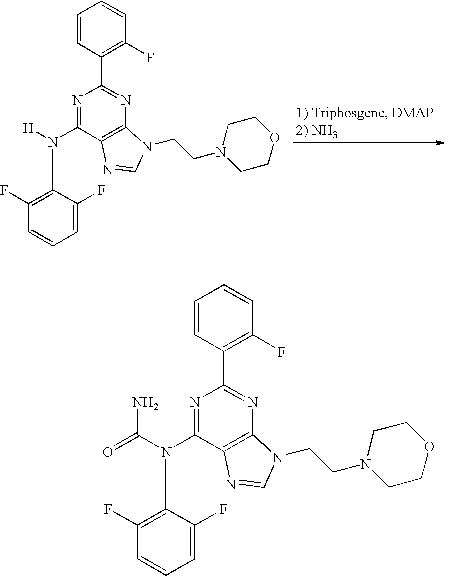

The compound of Example 21 (e) was reacted by the procedure of Example 24 to afford the title compound as a white solid. MS(ES) m/e 498 [M+H]⁺.

### Example 50

### Preparation of 1-(2,6-Difluorophenyl)-1-[2-(4-fluoro-2-methylphenyl)-7-methyl-7H-pyrrolo[2,3-d]pyrimidin-4-yl]urea

### a) 1,7-dihydro-pyrrolo[2,3-d]pyrimidine-2,4-dione

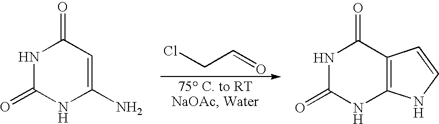

Prepared by the method described in Senda, et al., Chem. Pharm. Bull 1974, 22(7), 1459-1467, whose disclosure is incorporated herein by reference in its entirety.

### b) 2,4-dichloro-7H-pyrrolo[2,3-d]pyrimidine

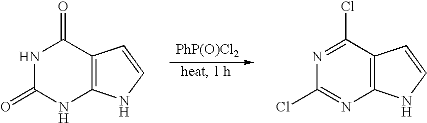

The compound of Example 50(a)(1.0 g., 0.0066 mol) was heated to 165° with phenylphosphonic dichloride (5 mL) with stirring under Ar to afford a dark viscous syrup, which was poured over crushed ice (100 g); triturated with a solution of CH₂Cl₄ (50 mL) and Et₂O (50 mL) and filtered through a Celite®521 mat. Layers of the filtrate were separated; the aqueous layer re-extracted with Et₂O (2×25 mL); combined organic layers were dried (Na₂SO₄) and filtered through a short silica gel column, with Et₂O to afford the title compound as a yellow solid (510 mg, 41%). MS(ES)m/e 186 [M−H]⁻, m/e 188 [M−H]⁻

### c) 2,4-Dichloro-7-methyl-7H-pyrrolo[2,3-d]pyrimidine

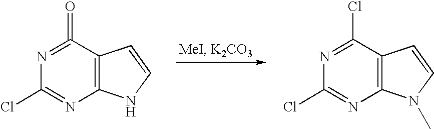

The compound of Example 50(b) (0.9 g, 0.0048 mol) was dissolved in dry DMF (13 mL), and with stirring under Ar, treated in turn with K₂CO₃ (anhydrous, powder) (1.314 g, 0.0095 mol) and CH₃I (0.684 g, 274 uL, 0.0048 mol). The mixture was stirred 16 h at 230, filtered; oncentrated, and partitioned between H₂O (30 mL) and Et₂O (4×30 mL). The combined Et₂O was dried (Na₂SO₄), then evaporated to afford the title compound, as a pale yellow solid (0.7 g, 72%). ¹H NMR (400 MHz, CDCl₃) δ 7.18 (d, 1H), 6.59 (d, 1H), 3.86 (s, 3H).

### d) 2-Chloro-7-methyl-4-methylsulfanyl-7H-pyrrolo[2,3-d]pyrimidine

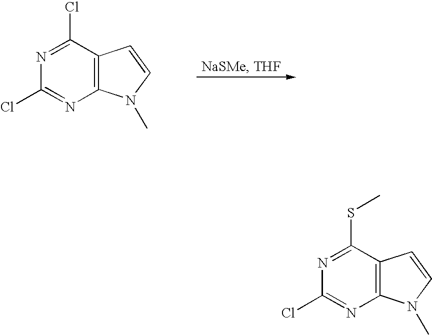

The pyrrolo[2,3-d]pyrimidine (2.02 g, 0.01 mol) of Example 50(c) in dry, redistilled, THF (120 mL) was treated with CH₃SNa (0.875 g, 0.0125 mol) and the mixture was stirred for 18 h, under Ar, at 23°, saturated aq NaCl (50 mL) was added; the layers were separated; the organic layer was washed with saturated aq NaCl; dried (Na₂SO₄); and concentrated to afford a orange brown solid which was dissolved in C₁-C₁₂, filtered through silica gel; to afford the title compound, a butter colored solid (1.79 g, 84.2%). ¹H NMR (400 MHz, CDCl₃) δ 7.03 (d, 1H), 6.50 (d, 1H), 3.83 (s, 3H), 2.71 (s, 3H).

### e) 2-(4-Fluoro-2-methylphenyl)-7-methyl-4-methylsulfanyl-7H-pyrrolo[2,3-d]pyrimidine

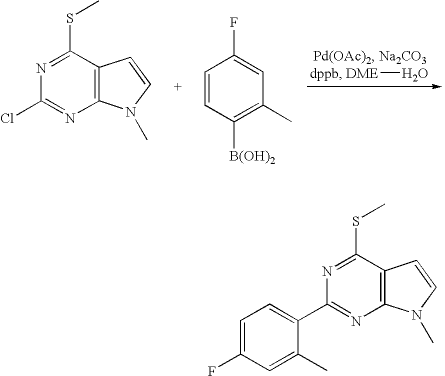

Pd(OAc)₂ (67 mg, 0.29 mmol) in ethylene glycol dimethyl ether (47 mL) was purged with Ar, then with stirring, under Ar, 1,4-bis(diphenylphosphino)butane (138 mg, 0.32 mmol) was added. The mixture was gently warmed to give an amber colored solution which was cooled to 23° over 0.5 h. Then in rapid succession, under Ar, was added the compound of Example 50(d) (1.26 g, 0.00587 mol), 2-methyl-4-fluorophenylboronic acid (0.99 g, 0.0065 mol), Na₂CO₃ (1.48 g, 0.0176 mol) and H₂O (2.9 mL). The mixture was stirred in a sealed vessel for 4 days at 130°, cooled to 23°; filtered; and concentrated to dryness. The residue was taken up in CH₂Cl₂ (7 mL), filtered and the filtrate applied to a Chromatotron™ plate for purification (silica gel, 2000 um thickness plate, 1% ethanol/hexane) to afford the title compound as a white solid (1.60 g, 95%). MS(FAB) m/e 288 [M+H]⁺.

### f) 2-(4-Fluoro-2-methylphenyl)-7-methyl-4-methanesufonyl-7H-pyrrolo[2,3-d]pyrimidine

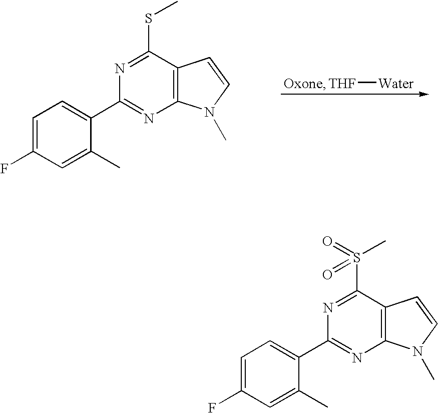

A solution of the compound of Example 50(e) (1.22 g, 0.0043 mol) in THF (60 mL) stirring at 0°, was treated, dropwise over 20 min, with a solution of Oxone® (4.3 g, 0.007 mol) in H₂O (40 mL), the resulting mixture was allowed to warm to 23° and stirred 16 h, and then was extracted with EtOAc (2×50 mL); and the organic extract was dried (NA₂SO₄) and evaporated to give the title compound as a yellow gum, which upon standing overnight crystallized (1.2 g, 87%) MS(FAB) m/e 320 [M+H]⁺.

### g) (2,6-difluorophenyl)-[2-(4-fluoro-2-methylphenyl)-7-methyl-7H-pyrrolo[2,3-d]pyrimidin-4-yl]amine

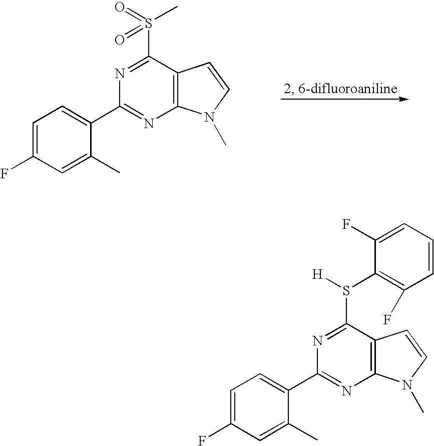

The compound of Example 50(f) (170 mg, 0.00053 mol) in 2,6-difluoroaniline (750 uL) was stirred for 18 h in a sealed vessel, under Ar, at 1650. Excess 2,6-difluoroaniline was removed in vacuo and the residue was taken up in CH₂Cl₂ (4 mL) and applied to a Chromatotron™ plate for purification (silica gel, 2000 um thickness plate, step gradient, 10-20% ethyl acetate/hexane) evaporation of the eluent afforded the title compound as a crystalline solid (72 mg, 37%) MS(FAB) m/e 369 [M+H]⁺.

### h) 1-(2,6-Difluorophenyl)-1-[2-(4-fluoro-2-methylphenyl)-7-methyl-7H-pyrrolo[2,3-d]pyrimidin-4-yl]urea

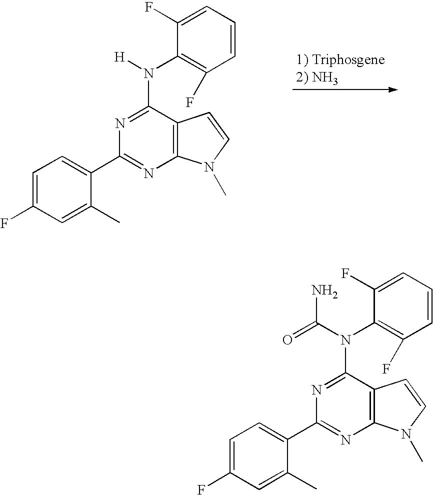

A solution of triphosgene (29 mg, 0.095 mmol) in CH₂Cl₂ (0.75 mL) stirring at 23° was treated slowly by syringe with a solution of the compound of Example 50(g) (36 mg, 0.1 mmol) and N,N-diisopropylethylamine (16 uL, 14.2 mg, 0.11 mmol) in C₁-C₁₂ (1.0 mL), over 1.5 min. The solution was stirred for 18 h, then gently a stream of NH₃ gas was introduced for 2 min. The viscous mixture stirred for 10 min; then evaporated to dryness; partitioned between CH₂Cl₂ (2×10 mL) and H₂O (2 mL); and the combined CH₂Cl₂ was dried (Na₂SO₄) and evaporated to a clear residue (35 mg). This was purified on a Chromatotron™ plate (silica gel, 1000 um thickness plate, step gradient, 0-1% methanol/methylene chloride) to afford the title compound (13 mg, 32%) MS(FAB) m/e 412 [M+H]⁺.

### Example 51

### Preparation of 1-(2,6-Difluorophenyl)-1-[2-(4-fluoro-2-methylphenyl)-7-methyl-7H-pyrrolo[2,3-d]pyrimidin-4-yl]-3-methylurea

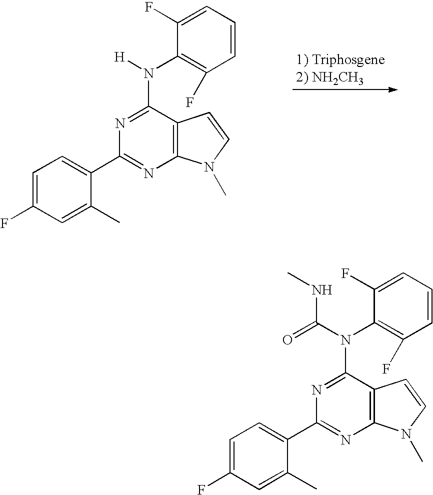

The compound of Example 50(g) (41 mg, 0.11 mmol) was reacted by the procedure of Example 50(h) except that NH₂CH₃ was used instead of NH₃ to afford the title compound (36.2 mg, 76%) MS(FAB) m/e 426 [M+H]⁺.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A compound of the formula: 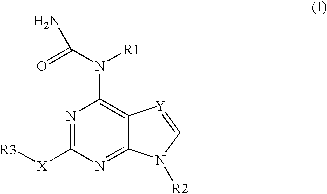
wherein
R₁ is hydrogen, C₁₋₁₀ alkyl, C₃₋₇ cycloalkyl, C₃₋₇ cycloalkyl alkyl, C₅₋₇ cycloalkenyl, C₅₋₇ cycloalkenylalkyl, aryl, arylalkyl, heterocyclic, heterocyclicalkyl, heteroaryl, or heteroarylalkyl moiety, all of which moieties may be optionally substituted;
R₂ is C₁₋₁₀ alkyl, C₃₋₇ cycloalkyl, C₃₋₇ cycloalkylalkyl, C₅₋₇ cycloalkenyl, C₅₋₇ cycloalkenylalkyl, aryl, arylC₁₋₁₀ alkyl, heteroaryl, heteroarylC₁₋₁₀ alkyl, heterocyclic, heterocyclylC₁₋₁₀ alkyl moiety, all of which moieties may be optionally substituted;
X is a bond, oxygen, nitrogen or sulfur;
R₃ is an optionally substituted aryl or optionally substituted heteroaryl moiety;
Y is carbon or nitrogen;
or a pharmaceutically acceptable salt thereof.

2. The compound according to claim 1 wherein R₁ is an optionally substituted aryl, arylalkyl, heterocylicalkyl, aminoalkyl, or mono- or di-substituted aminoalkyl.

3. The compound according to claim 2 wherein the aryl or arylalkyl is optionally substituted one or more times, independently by halogen, alkyl, hydroxy, alkoxy, amino, or halosubstituted alkyl.

4. The compound according to claim 3 wherein the aryl is phenyl substituted one or more times with halogen.

5. The compound according to claim 1 wherein R₂ is an optionally substituted alkyl, or heteroarylalkyl.

6. The compound according to claim 5 wherein the alkyl is an optionally substituted by hydroxy, C(O)OR₆, NR₄R₁₄.

7. The compound according to claim 1 wherein X is a bond.

8. The compound according to claim 7 wherein R₃ is an optionally substituted aryl.

9. The compound according to claim 8 wherein the aryl is optionally substitued 1 to 3 times independently by halogen, alkyl, amino, or hydroxy.

10. The compound according to claim 1 which is:
N-1-(2,6-Difluorophenyl)-N-[2-(4-fluoro-2-methylphenyl)-7-methyl-7H-pyrrolo[2,3-d]pyrimidin-4-yl]urea, or a pharmaceutically acceptable salt thereof.

11. A pharmaceutical composition comprising an effective amount of a compound according to claim 1 and a pharmaceutically acceptable carrier or diluent.

12. A method of treating a CSBP/RK/p38 kinase mediated disease in a mammal in need thereof, which method comprises administering to said mammal an effective amount of a compound of Formula (I) according to claim 1.

13. The method according to claim 12 wherein the CSBP/RK/p38 kinase mediated disease is psoriatic arthritis, Reiter's syndrome, gout, traumatic arthritis, rubella arthritis, acute synovitis, rheumatoid arthritis, rheumatoid spondylitis, osteoarthritis, gouty arthritis and other arthritic condition, sepsis, septic shock, endotoxic shock, gram negative sepsis, toxic shock syndrome, cerebral malaria, meningitis, ischemic and hemorrhagic stroke, neurotrauma/closed head injury, asthma, adult respiratory distress syndrome, chronic pulmonary inflammatory disease, chronic obstructive pulmonary disease, silicosis, pulmonary sarcososis, bone resorption disease, osteoporosis, restenosis, cardiac, brain and renal reperfusion injury, thrombosis, glomerularnephritis, chronic renal failure, diabetes, diabetic retinopathy, macular degeneration, graft vs. host reaction, allograft rejection, inflammatory bowel disease, Crohn's disease, ulcerative colitis, neurodegenrative disease, muscle degeneration, diabetic retinopathy, macular degeneration, tumor growth and metastasis, angiogenic disease, rhinovirus infection, eczema, contact dermatitis, psoriasis, sunburn, and conjunctivitis.

14. A compound of the formula: 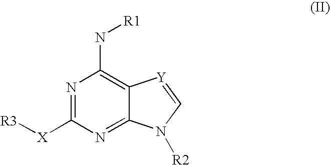
wherein
R₁ is hydrogen, C₁₋₁₀ alkyl, C₃₋₇ cycloalkyl, C₃₋₇ cycloalkyl alkyl, C₅₋₇ cycloalkenyl, C₅₋₇ cycloalkenylalkyl, aryl, arylalkyl, heterocyclic, heterocyclicalkyl, heteroaryl, or heteroarylalkyl moiety, all of which moieties may be optionally substituted;
R₂ is C₁₋₁₀ alkyl, C₃₋₇ cycloalkyl, C₃₋₇ cycloalkylalkyl, C₅₋₇ cycloalkenyl, C₅₋₇ cycloalkenylalkyl, aryl, arylC₁₋₁₀ alkyl, heteroaryl, heteroarylC₁₋₁₀ alkyl, heterocyclic, heterocyclylC₁₋₁₀ alkyl moiety, all of which moieties may be optionally substituted;
X is a bond, oxygen, nitrogen or sulfur;
R₃ is an optionally substituted aryl or optionally substituted heteroaryl moiety;
Y is carbon or nitrogen;
or a pharmaceutically acceptable salt thereof.

15. The compound according to claim 14 wherein R₁ is an optionally substituted aryl, arylalkyl, heterocylicalkyl, aminoalkyl, or mono- or di-substituted aminoalkyl.

16. The compound according to claim 15 wherein the aryl or arylalkyl is optionally substituted one or more times, independently by halogen, alkyl, hydroxy, alkoxy, amino, or halosubstituted alkyl.

17. The compound according to claim 16 wherein the aryl is phenyl substituted one or more times with halogen.

18. The compound according to claim 14 wherein R₂ is an optionally substituted alkyl, or heteroarylalkyl.

19. The compound according to claim 18 wherein the alkyl is an optionally substituted by hydroxy, C(O)OR₆, NR₄R₁₄.

20. The compound according to claim 14 wherein X is a bond.

21. The compound according to claim 20 wherein R₃ is an optionally substituted aryl.

22. The compound according to claim 21 wherein the aryl is optionally substitued 1 to 3 times independently by halogen, alkyl, amino, or hydroxy.

23. A pharmaceutical composition comprising an effective amount of a compound according claim 14 and a pharmaceutically acceptable carrier or diluent.

24. A method of treating a CSBP/RK/p38 kinase mediated disease in a mammal in need thereof, which method comprises administering to said mammal an effective amount of a compound of Formula (I) according to claim 14.

25. The method according to claim 24 wherein the CSBP/RK/p38 kinase mediated disease is psoriatic arthritis, Reiter's syndrome, gout, traumatic arthritis, rubella arthritis, acute synovitis, rheumatoid arthritis, rheumatoid spondylitis, osteoarthritis, gouty arthritis and other arthritic condition, sepsis, septic shock, endotoxic shock, gram negative sepsis, toxic shock syndrome, cerebral malaria, meningitis, ischemic and hemorrhagic stroke, neurotrauma/closed head injury, asthma, adult respiratory distress syndrome, chronic pulmonary inflammatory disease, chronic obstructive pulmonary disease, silicosis, pulmonary sarcososis, bone resorption disease, osteoporosis, restenosis, cardiac, brain and renal reperfusion injury, thrombosis, glomerularnephritis, chronic renal failure, diabetes, diabetic retinopathy, macular degeneration, graft vs. host reaction, allograft rejection, inflammatory bowel disease, Crohn's disease, ulcerative colitis, neurodegenrative disease, muscle degeneration, diabetic retinopathy, macular degeneration, tumor growth and metastasis, angiogenic disease, rhinovirus infection, eczema, contact dermatitis, psoriasis, sunburn, and conjunctivitis.

26. A process of making a compound of Formula (I) according to claim 1 which comprises reacting a compound of Formula (II), according to claim 14 with tri-phosgene, phosgene, diphenyl carbonate or other activated carbonate equivalents, and ammonia to yield a compound of Formula (I).
